(19) United States
(12) Patent Application Publication
(10) Pub. No.: US 2007/0036831 A1
(43) Pub. Date: Feb. 15, 2007

(54) Nanoemulsion compositions having anti-inflammatory activity

(75) Inventor: James R. Baker, Ann Arbor, MI (US)

Correspondence Address: FOLEY AND LARDNER LLP;SUITE 500, 3000 K STREET NW, WASHINGTON, DC, 20007, US

(73) Assignee: NanoBio Corporation

(21) Appl. No.: 11/501,007

(22) Filed: Aug. 9, 2006

Related U.S. Application Data
(60) Provisional application No. 60/706,429, filed on Aug. 9, 2005.

(51) Int. Cl. A61K 31/573; A61K 9/00

(52) U.S. Cl. 424/400; 514/171; 977/906

(57) ABSTRACT

Nanoemulsion compositions with low toxicity that demonstrate broad spectrum inactivation of microorganisms or prevention of diseases are described. The nanoemulsions contain an aqueous phase, an oil phase comprising an oil and an organic solvent, at least one anti-inflammatory agent, and one or more surfactants. Methods of making nanoemulsions and inactivating pathogenic microorganisms are also provided.

### FIELD OF THE INVENTION

The present disclosure relates to compositions and methods for prevention and treatment of infection by variety of pathogenic microorganisms.

### BACKGROUND OF THE INVENTION

Effective treatment of infections, including bacterial and viral infections, can involve treatment of the primary infection as well as secondary symptoms of that infection. Such treatment includes eradication of the pathogenic infection in combination with inhibition of the inflammation process, allowing damaged and inflamed tissues to heal.

To effectively treat a pathogenic microbial infection, the microbial source of the infection should be eliminated. Although antibiotic and antimicrobial therapy is very effective and a mainstay of modern medicine, these therapies suffer from several disadvantages. For example, bacterial strains can develop antibiotic resistance. A person infected with an antibiotic resistant strain of bacteria faces serious and potentially life-threatening consequences because antibiotics cannot eliminate the infection. Pneumococci, which cause pneumonia and meningitis, Salmonella and E. coli which cause diarrhea, and enterococci which cause blood stream, surgical wound, and urinary tract infections can all develop antibiotic resistance resulting in fatal infections.

Moreover, antibiotics are not effective in eliminating or inactivating bacterial spores and viruses. Bacteria of the Bacillus genus and others form stable spores that resist harsh conditions and extreme temperatures. For example, contamination of farmlands with B. anthracis can lead to a fatal disease in domestic, agricultural, and wild animals, as well as in humans in contact with infected animals or animal products. B. anthracis infection in humans is no longer common due to effective animal controls that include vaccines, antibiotics, and appropriate disposal of infected livestock. However, animal anthrax infection still represents a significant problem due to the difficulty of decontaminating land and farms. Moreover, B. anthracis spores can be used as a biological weapon. Other members of the Bacillus genus are also reported to be etiological agents for many human diseases. B. cereus is a common pathogen involved in food borne diseases due to the ability of the spores to survive cooking procedures. It is also associated with local sepsis, wound and systemic infection. Disinfectants and biocides, such as sodium hypochlorite, formaldehyde and phenols can be effective against bacterial spores, but are not well suited for treatment of humans and other animals. The toxicity of these compounds can result in tissue necrosis and severe pulmonary injury following contact or inhalation of volatile fumes.

Viruses are additional pathogens that infect human and animals which currently lack effective means of inactivation. For example, influenza A virus is a common respiratory pathogen widely used as a model system to test anti-viral agents in vitro and in vivo. The envelope glycoproteins of influenza A, hemagglutinin (HA) and neuraminidase (NA), which determine the antigenic specificity of viral subtypes, mutate readily, rendering antibodies incapable of neutralizing the virus. Current anti-viral compounds and neuraminidase inhibitors are minimally effective and viral resistance is common.

It is desirable to use a two-fold microbial infection treatment regimen involving the use of a broad spectrum antimicrobial compositions as well as a composition having anti-inflammatory activity.

### SUMMARY OF THE INVENTION

Accordingly, there remains a need in the art for anti-microbial compositions capable of inactivating microorganisms and providing anti-inflammatory activity while minimizing microbial resistance and toxicity to the recipient.

To address these and other needs, emulsions comprising an aqueous phase, an oil phase comprising an oil and an organic solvent, at least one anti-inflammatory agent, and at least one surfactant. The emulsion comprises particles preferably having an average diameter of less than or equal to about 250 nm.

In one embodiment, the invention provides a method of reducing the average nanoemulsion particle size of a composition comprising a nanoemulsion, comprising treating a nanoemulsion comprising an aqueous phase, an oil phase comprising an oil and an organic solvent, at least one anti-inflammatory agent, and a surfactant, and having nanoemulsion particles of an average diameter of greater than or equal to about 250 nm, so as to reduce the average diameter of the nanoemulsion particles to less than or equal to about 250 nm.

In another embodiment, the invention provides a method of making a nanoemulsion, comprising passing a first nanoemulsion through a high pressure homogenizer or a microfluidizer under conditions effective to reduce the average diameter of the nanoemulsion particles less than or equal to about 250 nm. The nanoemulsion comprises an aqueous phase, an oil phase comprising an oil and an organic solvent, at least one anti-inflammatory agent, and one or more surfactants. The nanoemulsion particles have an average diameter of greater than or equal to about 250 nm.

A further embodiment provides a method of inactivating a microorganism, comprising contacting the microorganism with a composition comprising a nanoemulsion for a time effective to inactivate the microorganism. The nanoemulsion comprises an aqueous phase; an oil phase comprising an oil and an organic solvent, at least one anti-inflammatory agent, and one or more surfactants. The nanoemulsion particles have an average diameter of less than or equal to about 250 nm.

Yet another embodiment provides a method of inactivating a pathogenic microorganism comprising contacting a subject infected with the microorganism with a composition comprising a nanoemulsion. The nanoemulsion comprises an aqueous phase, an oil phase comprising an oil and an organic solvent, at least one anti-inflammatory agent, and one or more surfactants, wherein the nanoemulsion comprises particles having an average diameter of less than or equal to about 250 nm.

Another embodiment provides a method of preventing an infected state caused by a microorganism, comprising administering to a subject, either before or after exposure to a microorganism, a composition comprising a nanoemulsion. The nanoemulsion comprises an aqueous phase, an oil phase comprising an oil and an organic solvent, at least one anti-inflammatory agent, and one or more surfactants, wherein the nanoemulsion comprises particles having an average diameter of less than or equal to about 250 nm.

The invention further provides a kit comprising a composition comprising a nanoemulsion composition having anti-inflammatory activity, wherein the composition is provided in a single formulation or a binary formulation, wherein the binary formulation is mixed prior to using the composition.

The above described and other features are exemplified by the following FIGURES and detailed description.

### BRIEF DESCRIPTION OF THE DRAWINGS

FIG. 1. Average separation of neat (100%) emulsions stored at 55° C.

FIG. 2. Average settling of 10% emulsions stored at 55° C.

FIG. 3. Average settling of 2.5% emulsions stored at 55° C.

FIG. 4. Change in pH after accelerated stability testing. pH of neat and diluted emulsions is measured on day 0 and after 31 days incubation at 55° C.

FIG. 5. Dependence of nanoemulsion particle size of passage number and pressure in Avestin EmulsiFlex® C3.

### DETAILED DESCRIPTION OF THE PREFERRED EMBODIMENTS

Compositions having emulsion particles with an average particle diameter of less than or equal to about 250 nm (“small particle size nanoemulsion”) have improved stability and/or activity. Such compositions further having anti-inflammatory activity are particularly well suited for the treatment of microbial infections. These small particle size nanoemulsions are useful in a wide range of applications for decreasing the infectivity, morbidity, and/or rate of mortality associated with a variety of pathogenic microorganisms. Anti-inflammatory activity, in conjunction with the anti-microbial activity can eliminate a microbial infections and speed healing of tissues. As used herein, the term “pathogenic microorganism” refers to a biological microorganism that is capable of producing an undesirable effect upon a host animal, and includes, for example, without limitation, bacteria, viruses, bacterial spores, molds, mildews, fungi, and the like. This includes all such biological microorganisms, regardless of their origin or of their method of production, and regardless of whether they exist in facilities, in munitions, weapons, or elsewhere.

Small particle size nanoemulsion compositions having anti-inflammatory activity are useful, for example, as therapeutics for humans or animals, for decontaminating individuals colonized or otherwise infected by pathogenic microorganisms, for prophylaxis, treatment, and decreasing the infectivity of pathogenic microorganisms. The inactivation of a broad range of pathogenic microrganisms, including, for example, vegetative bacteria and enveloped viruses and bacterial spores, combined with low toxicity, make small particle size nanoemulsions well-suited for use as a general decontamination agent before a specific pathogen is identified. Moreover, the anti-inflammatory activity of these compositions facilitates tissue healing.

A. Nanoemulsion Compositions

Particle size reduction to produce a small particle size nanoemulsion from a standard emulsion is efficiently and economically accomplished by high-pressure homogenizer or microfluidizer. Small particle size nanoemulsions can be rapidly produced in large quantities and are stable for many months at a broad range of temperatures.

An emulsion is a composition containing an aqueous phase and an oil phase. The term “emulsion” refers to, without limitation, any oil-in-water dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Classical or standard emulsions comprise lipid structures having an average particle size of greater than about 5 μm in diameter. Standard nanomulsions having smaller particle sizes are known, and comprise lipid structures having an average particle diameter of about 500 nm to about 5 μm. In one embodiment, a standard nanoemulsion has an average particle size of about As used herein, “small particle size nanoemulsions” refers to emulsions having an average particle diameters of less than or equal to about 250 nm. In one embodiment, average particle diameter is less than or equal to about 200 nm, less than or equal to about 150 nm, less than or equal to about 100 nm, or less than or equal to about 0.50 nm. As used herein, the term “nanoemulsion” can encompass both standard and small particle size nanoemulsions.

Emulsion particle size can be determined using any means known in the art, such as, for example, using laser light scattering.

A nanoemulsion composition contains about 5 to about 50 percent by volume (vol %) of aqueous phase. As used herein, percent by volume (vol %) is based on the total volume of an emulsion or small particle size nanoemulsion. In one embodiment, the aqueous phase is about 10 to about 40 vol %. In another embodiment, the aqueous phase is about 15 to about 30 vol %. The aqueous phase ranges from a pH of about 4 to about 10. In one embodiment the pH of the aqueous phase ranges from about 6 to about 8. The pH of the aqueous phase can be adjusted by addition of an acid or a base such as, for example, hydrochloric acid or sodium hydroxide. In one embodiment, the aqueous phase is deionized water (hereinafter “diH₂O”) or distilled water.

The oil phase of a nanoemulsion contains an oil and an organic solvent. The oil phase of a nanoemulsion contains about 30 to about 90 vol % oil, based on the total volume of the nanoemulsion. In one embodiment, the nanoemulsion contains about 60 to about 80 vol % oil. In another embodiment, the nanoemulsion contains about 60 to about 70 vol % oil. The oil phase also contains from about 3 to about 15 vol % of an organic solvent based on the total volume of the nanoemulsion. In one embodiment, the nanoemulsion contains about 5 to about 10 vol % of an organic solvent.

Suitable oils include, but are not limited to, soybean oil, avocado oil, squalene oil, olive oil, canola oil, corn oil, rapeseed oil, safflower oil, sunflower oil, fish oils, cinnamon bark, coconut oil, cottonseed oil, flaxseed oil, pine needle oil, silicon oil, mineral oil, essential oil, flavor oils, water insoluble vitamins, and combinations comprising one or more of the foregoing oils. In one embodiment, the oil is soybean oil.

Suitable organic solvents include, but are not limited to, organic phosphate solvents, alcohols, and combinations comprising one or more of the foregoing solvents. Suitable organic phosphate solvents include, but are not limited to, dialkyl and trialkyl phosphates having one to ten carbon atoms, more preferably two to eight carbon atoms. The alkyl groups of the di- or trialkyl phosphate can all the same or the alkyl groups can be different. In one embodiment, the trialkyl phosphate is tri-n-butyl phosphate. Without being held to theory, it is believed that organic solvents used in the small particle size nanoemulsions serve to stabilize the nanoemulsion and remove or disrupt the lipids in the membranes of pathogens.

Suitable alcohols include, for example, C₁-C₁₂ alcohols, diols, and triols, for example glycerol, methanol, ethanol, propanol, octanol, and combinations comprising one or more of the foregoing alcohols. In one embodiment, the alcohol is ethanol or glycerol, or a combinations thereof.

Small particle size nanoemulsion compositions can also contain one or more surfactants, present in the aqueous phase, the oil phase, or both phases of a nanoemulsion. While not limited to any particular proposed mechanism, a nanoemulsion composition may function to remove proteins from bacterial membranes, such that a surfactant that will “strip” a membrane of its proteins may be useful. A nanoemulsion can contain about 3 to about 15 vol % of surfactant, based on the total volume of nanoemulsion. In one embodiment, the nanoemulsion contains about 5 to about 10 vol % of surfactant.

Suitable surfactants include, but are not limited to, a variety of ionic and nonionic surfactants, as well as other emulsifiers capable of promoting the formation of nanoemulsions. Surfactants that allow the oil phase to remain suspended in the water phase can be used. In one embodiment, the nanoemulsion comprises a non-ionic surfactant such as a polysorbate surfactant, i.e., polyoxyethylene ether. Other useful surfactants include, but are not limited to, the polysorbate detergents sold under the tradenames TWEEN® 20, TWEEN® 40, TWEEN® 60, TWEEN® 80, phenoxypolyethoxyethanols and polymers thereof, such as Triton® (i.e., X-100, X-301, X-165, X-102, X-200), Poloxamer® 407, Spans (20, 40, 60, and 80), tyloxapol, and combinations comprising one or more of the foregoing surfactants. Additional appropriate surfactants include Brij®30, Brij®35, Brij®52, Brij®56, Brij®58, Brij®72, Brij®76, Brij®78, Brij®92, Brij®97, Brij®98, and Brij® 700. Anionic surfactants include, but are not limited to sodium dodecyl sulfate (SDS). Mixtures of surfactants are also contemplated. In one embodiment, the surfactant is TWEEN® 20 or Triton® X-100 or a combination thereof. Triton X-100 is a strong non-ionic detergent and dispersing agent widely used to extract lipids and proteins from biological structures. It also has virucidal effect against a broad spectrum of enveloped viruses. In another embodiment, the surfactant is nonoxynol-9.

Suitable anti-inflammatory agents include steroidal and non-steroidal anti-inflammatory agents. Any suitable steroid can be used. In one embodiment, a nanoemulsion composition can include one or more steroids classified as very potent, potent, moderately potent, or mild. Very potent steroids include, for example, betamethasone dipropionate (Diprolene), clobetasol 17-Propionate (Dermovate), halobetasolpropionate (Ultravate), Halcinonide (Halog). Potent steroids include, for example, amcinonide (Cyclocort), betamethasone dipropionate (Diprolene, generics), betamethasone valerate (Betaderm, Belestoderm,Prevex), Desoximetasone (Desoxi,Topicort), diflucortolone valerate (Nerisone), fluocinonlone acetonide (Derma,Fluoderm,Synalar), fluocinonide (Lidemol, Lidex, Tyderm, Tiamol, Topsyn), and mometasone furoate. Moderately potent steroids include, for example, betamethasone valerate (Betnovate), betamethasone valerate (Celestoderm), clobetasone 17-butyrate (Eumovate), desonide (Desocort), hydrocortisone acetate (Cortef, Hyderm),hydrocortisone valerate (Westcort, Hydroval), prednicarbate (Dermatop), triamcinolone acetonide (Kenalog,Traiderm). Mild steroids include, for example, loratodine (Claratin) desonide (Desocort), hydrocortisone (Cortate, Cortoderm), hydrocortisone acetate (Cortef, Hyderm), or a combination thereof.

Any suitable non-steroidal anti-inflammatory drug can be used. In one embodiment, the non-steroidal anti-inflammatory drug can be, for example, aspirin (Anacin, Ascriptin, Bayer, Bufferin, Ecotrin, Excedrin), choline and magnesium salicylates (CMT, Tricosal, Trilisate), choline salicylate (Arthropan), celecoxib (Celebrex), diclofenac potassium (Cataflam),diclofenac sodium (Voltaren, Voltaren XR), diclofenac sodium with misoprostol (Arthrotec), diflunisal (Dolobid), etodolac (Lodine, Lodine XL), fenoprofen calcium (Nalfon), flurbiprofen (Ansaid), ibuprofen (Advil, Motrin, Motrin IB, Nuprin), indomethacin (Indocin, Indocin SR), ketoprofen (Actron, Orudis, Orudis KT, Oruvail), magnesium salicylate (Arthritab, Bayer Select, Doan's Pills, Magan, Mobidin, Mobogesic), meclofenamate sodium (Meclomen), mefenamic acid (Ponstel), meloxicam (Mobic), nabumetone (Relafen), naproxen (Naprosyn, Naprelan), naproxen sodium (Aleve, Anaprox), oxaprozin (Daypro), piroxicam (Feldene), rofecoxib (Vioxx), salsalate (Amigesic, Anaflex 750, Disalcid, Marthritic, Mono-Gesic, Salflex, Salsitab), sodium salicylate, sulindac (Clinoril), tolmetin sodium (Tolectin), valdecoxib (Bextra), or a combination thereof.

Any suitable concentration of anti-inflammatory agent can be used. For example, steroid concentration can be from 0.01 to 10%. In one embodiment, steroid concentration can be from approxiamtely 0.05 to approximately 1%. In another embodiment, steroid concentration can be less than approximately 10%, less than approximately 5%, less than approximately 3%, less than approximately 2%, less than approximately 1%, less than 0.5%, less than 0.5%, less than 0.2%, less than 0.1%, or less than approximately 0.05%.

Nanoemulsion compositions can further contain various additives. Exemplary additives include, for example, activity modulators, gelling agents, thickeners, auxiliary surfactants, other agents that augment cleaning and aesthetics, and combinations comprising at least one of the foregoing, so long as they do not significantly adversely affect the activity and/or stability of the emulsions. Additives can be incorporated into the nanoemulsion or formulated separately from the nanoemulsion, i.e., as a part of a composition containing a nanoemulsion.

“Activity modulators” are additives that affect the activity of a nanoemulsion against the target microorganism. Exemplary activity modulators are interaction enhancers such as germination enhancers, therapeutic agents, buffers, and the like, which are described below.

One class of activity modulators thus includes “interaction enhancers,” compounds, or compositions that increase the interaction of the nanoemulsion with the cell wall of a bacterium (e.g., a Gram positive or a Gram negative bacteria) or a fungus, or with a virus envelope. Again, without being bound by theory, it is proposed that the activity of the emulsions is due, in part, to the interaction of a nanoemulsion with a microorganism membrane or envelope. Suitable interaction enhancers include compounds that increase the interaction of the nanoemulsion with the cell wall of Gram negative bacteria such as Vibrio, Salmonella, Shigella, Pseudomonas, Escherichia, Klebsiella, Proteus, Enterobacter, Serratia, Moraxella, Legionella, Bordetella, Helicobacter, Haemophilus, Neisseria, Brucella, Yersinia, Pasteurella, Bacteiods, and the like.

One exemplary interaction enhancer is a chelating agent. Suitable chelating agents include ethylenediaminetetraacetic acid (EDTA), ethylenebis(oxyethylenenitrilo)tetraacetic acid (EGTA), and combinations thereof. Chelating agents can be prepared in water or in a buffer, such as, for example, TRIS buffer. Chelating agents can be premixed with the aqueous phase or can be added to a diluent. Chelating agents can be used at a concentration of about 1 μM to about 50 mM, based on the total volume of the nanoemulsion composition. In one embodiment, the concentration of the chelating agent is between about 100 μM to about 50 mM. In a further embodiment, the concentration of chelating agent can be greater than or equal to about 25 μM, greater than or equal to about 50 μM, greater than or equal to about 70 μM greater than or equal to about 80 μM, greater than or equal to about 100 μM, greater than or equal to about 1 mM, or greater than or equal to about 2 mM. In an additional embodiment, the concentration of chelating agent can be less than or equal to about 40 mM, less than or equal to about 27 mM, less than or equal to about 25 mM, less than or equal to about 10 mM, or less than or equal to about 5 mM.

Another exemplary interaction enhancer is a cationic halogen-containing compound. A cationic halogen-containing compound can be premixed with the aqueous phase, or it may can be provided in combination with a nanoemulsion in a distinct formulation. A cationic halogen-containing compound can be used at a concentration of about 0.5 to about 7 vol. %, based on the total volume of the nanoemulsion. In one embodiment, a cationic halogen-containing compound can be used at a concentration of about 0.5 to about 3 vol. %, based on the total volume of the nanoemulsion.

Suitable cationic halogen-containing compounds include, but are not limited to, cetylpyridinium halides, cetyltrimethylammonium halides, cetyldimethylethylammonium halides, cetyldimethylbenzylammonium halides, cetyltributylphosphonium halides, dodecyltrimethylammonium halides, tetradecyltrimethylammonium halides, alkylbenzyldimethylammonium salts and combinations comprising one or more of the foregoing compounds. Suitable halides in the cationic halogen-containing compounds include chloride, fluoride, bromide and iodide. In one embodiment, the halide is chloride or bromide. In another embodiment the cationic halogen-containing compound is cetylpyridinium chloride or benzalkonium chloride or a combination thereof.

A “germination enhancer” enhances the germination of, for example, spores. Suitable germination enhancing agents include nucleosides, α-amino acids, salts and combinations thereof. Useful nucleosides include inosine. Useful α-amino acids include, for example, glycine and the L-enantiomers of alanine, valine, leucine, isoleucine, serine, threonine, lysine, phenylalanine, tyrosine, and the alkyl esters thereof. Suitable salts, include, for example, sodium chloride, ammonium chloride, magnesium chloride, calcium chloride, phosphate buffered saline (PBS), and potassium chloride. In one embodiment, the germination enhancer is a mixture of glucose, fructose, asparagine, sodium chloride, ammonium chloride, calcium chloride, and potassium chloride. In another embodiment, the germination enhancer is a combination containing L-alanine, inosine, PBS, and ammonium chloride.

Certain growth media contain germination enhancers and buffers. Thus, when testing nanoemulsions for their ability to inactivate spores, addition of germination enhancers may not be required, if the tests are conducted using media containing such germination enhancers. Similarly, the addition of certain growth media to emulsions can enhance sporicidal activity.

An effective amount of germination enhancer can be readily determined by one of ordinary skill in the art. Nucleosides and amino acids can be used in amounts of about 0.5 mM to about 100 mM. In one embodiment, nucleosides and amino acids are used at a concentration of about 1 mM to about 50 mM. In another embodiment, nucleosides and amino acids are used at a concentration of about 0.5 mM to about 5 mM. Salts can be present in amounts of about 0.5 mM to about 100 mM and PBS can be used at concentrations of about 0.05× to about 1×.

A germination enhancer can be incorporated into the aqueous phase prior to formation of the nanoemulsion. In one embodiment a germination enhancer is active at approximately neutral pH. In another embodiment, a germination enhancer can be active between pH of about 6 to about 8. Adjustment of pH of a nanoemulsion composition containing a germination enhancer can be achieved by any suitable means, such as, for example, dilution of a nanoemulsions in PBS or by preparations of a neutral nanoemulsion or by the addition of hydrochloric acid or sodium hydroxide.

“Therapeutic agent” refers to an agent that decreases the infectivity, morbidity, and/or rate of mortality associated with a pathogenic microorganism when administered to a subject affected by a pathogenic microorganism. Suitable therapeutic agents include, for example, antimicrobial agents, antiviral agents, antifungal agents, and the like, and combinations comprising one or more of the foregoing agents. There are many antimicrobial agents currently available for use in treating bacterial, fungal and viral infections. Generally, these agents include agents that inhibit cell wall synthesis (e.g., penicillins, cephalosporins, cycloserine, vancomycin, bacitracin), imidazole antifungal agents (e.g., miconazole, ketoconazole and clotrimazole), agents that act directly to disrupt the cell membrane of the microorganism (e.g., polymyxin and colistimethate and the antifungals nystatin and amphotericin B), agents that affect the ribosomal subunits to inhibit protein synthesis (e.g. chloramphenicol, the tetracyclines, erythromycin and clindamycin), agents that alter protein synthesis and lead to cell death (e.g. aminoglycosides), agents that affect nucleic acid metabolism (e.g. the rifamycins and the quinolones), antimetabolites (e.g., trimethoprim and sulfonamides), and the nucleic acid analogues (e.g. zidovudine, gangcyclovir, vidarabine, and acyclovir) which act to inhibit viral enzymes essential for DNA synthesis. Other useful therapeutic agents include, but are not limited to antimicrobials such as phenylphenol, propyl paraben and poly(hexamethylene biguanide) hydrochloride (PHMB).

Optionally, nanoemulsion compositions can be formed into gels by adding a gelling agent. Suitable gelling agents include, for example, hydrogels such as, for example, Natrosol® 250H NF (Hercules, Inc. Wilmington, Del.). A hydrogel can be added at concentration of about 0.5 wt % to about 5 wt %, based on the total volume of the gel. Other suitable gelling agents include, but are not limited to, about 0.05 wt % to about 3 wt % cellulose polymer, such as cellulose gum or cationic guar derivatives, and up to about 10 wt % petrolatum, glycerin, polyethylene glycol, incroquat behenyl TMS, cetyl palmitate, glycerol stearate, and the like.

A variety of auxiliary surfactants can optionally be used to enhance the properties of a nanoemulsion composition. The choice of auxiliary surfactant depends on the desire of the user with regard to the intended purpose of the composition and the commercial availability of the surfactant. In one embodiment, the auxiliary surfactant is an organic, water-soluble surfactant.

Other optional additives such as perfumes, brighteners, enzymes, colorants, detergent builders, suds suppressors, and the like can also be used in the compositions to enhance aesthetics and/or cleaning performance. Detergent builders sequester calcium and magnesium ions that might otherwise bind with and render less effective the auxiliary surfactants or co-surfactants. Detergent builders are particularly useful when auxiliary surfactants are used, and when the compositions are diluted prior to use with hard tap water, especially water having a hardness of, above about 12 grains/gallon.

A nanoemulsion composition can contain a suds suppressor. A suds suppressor is a low-foaming co-surfactant that prevents excessive sudsing during employment of the compositions on hard surfaces. Suds suppressors are also useful in formulations for no-rinse application of the composition. Concentrations of about 0.5 vol % to about 5 vol % are generally effective. Selection of a suds suppressor depends on its ability to formulate in a nanoemulsion composition and the residue as well as the cleaning profile of the composition. The suds suppressor should be chemically compatible with the components in a nanoemulsion composition and functional at the pH of a given composition. In one embodiment the suds suppressor or composition containing a suds suppressor does not leave a visible residue on surfaces on which a composition is applied.

Low-foaming co-surfactants can be used as a suds suppressor to mediate the suds profile in a nanoemulsion composition. Examples of suitable suds suppressors include block copolymers, alkylated primary and secondary alcohols, and silicone-based materials. Exemplary block co-polymers include, e.g., Pluronic® and Tetronic® (BASF Company). Alkylated alcohols include those which are ethoxylated and propoxylated, such as, tergitol (Union Carbide) or poly-tergent® (Olin Corp.). Silicone-based materials include DSE (Dow Corning). The suds suppressors can be incorporated into the composition by any means known in the art.

B. Method of Making Small Particle Size Nanoemulsions

Small particle size nanoemulsions and compositions containing small particle size nanoemulsions with anti-inflammatory activity can be produced by any suitable means. A small particle size nanoemulsion can be formed in the first instance or can be formed from a nanoemulsion having larger particles. For example, a small particle size nanoemulsion can be produced by reducing the particle size of a classical or standard nanoemulsion (hereinafter “standard nanoemulsion”), to produce a small particle size nanoemulsion wherein the average nanoemulsion particle size is less than about 250 nm. In other words, a nanoemulsion having an average particle diameter of greater than about 250 nm is treated in a manner effective to produce particles having an average diameter of less than or equal to about 250 nm. In one embodiment, small particle size nanoemulsion particles have an average diameter of less than or equal to about 200 nm, less than or equal to about 150 nm, less than or equal to about 100 nm, and less than or equal to about 50 nm.

Methods for the production of a standard nanoemulsion by mixing an oil phase with an aqueous phase are well-known. A nanoemulsion can be formed by blending an oil phase with an aqueous phase on a volume-to-volume basis ranging from about 1:9 to about 5:1, about 5:1 to about 3:1, or about 4:1, oil phase to aqueous phase. The oil and aqueous phases can be blended using an apparatus capable of producing shear forces sufficient to form a nanoemulsion such as, for example, a French press or a commercial low shear or high shear mixer. In one embodiment, the standard emulsions are prepared under conditions of high shear to produce a nanoemulsion having a substantially uniform particle size distribution. In one embodiment, a standard nanoemulsion for use in preparing a nanoemulsion composition is comprised of particles having an average diameter of about 500 nm to about 5 μm, about 500 nm to about 1 μm, 400 nm to about 5 μm, 400 nm to about 1 μm, from about 250 nm to about 5 μm, and from about 250 nm to about 1 μm. To obtain the desired pH, the pH of the aqueous phase can be adjusted using hydrochloric acid or sodium hydroxide.

Forming a small particle size nanoemulsion from a standard nanoemulsion can be accomplished, for example, by passing the standard nanoemulsion though a microfluidizer (Microfluidics Corp., Newton, Mass.) several times at a pressure sufficient to produce a desired particle size. A microfluidizer is a homogenizer that operates by pumping a fluid stream into an interaction chamber. The interaction chamber contains fixed-geometry microchannels that accelerate the fluid stream, resulting in high turbulence, shear, and cavitation. A H230Z (chamber 400 μm upstream of H210Z chamber (200 μm) can be used. Other chamber size and configurations (Y or Z) can be used in forming a nanoemulsion using a microfluidizer. During homogenization, a nanoemulsion can be circulated through a heat exchanger coil or otherwise cooled to keep the temperature of the nanoemulsion from increasing significantly. In one embodiment, a standard nanoemulsion is passed though the microfluidizer for two to five passes at a pressure of about 2,000 to about 10,000 psi. In another embodiment, the pressure is from 3,000 to about 4,000 pounds per square inch. These conditions can vary depending on factors such as standard nanoemulsion particle size, nanoemulsion composition, and desired final particle size

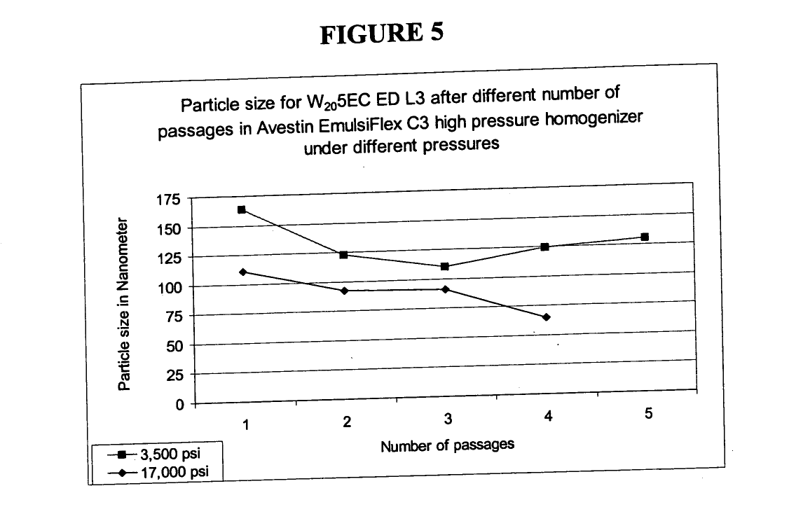

Another means of forming a small particle size nanoemulsion is passage of a standard nanoemulsion through a high pressure homogenizer, like an EmulsiFlex® high pressure homogenizer (Avestin, Inc., Ottawa, Canada). The number of passages through the homogenizer as well as the flow rate will depend on the particle size of the standard nanoemulsion, nanoemulsion composition, and the desired particle size of the resulting small particle size nanoemulsion. Operating pressure is independent from flow rate and will remain at the set value over the process time. In one embodiment, the operating pressure is from about 2,500 to about 20,000 psi. As with the microfluidizing method discussed above, a nanoemulsion can be cooled using a heat exchanger or other method and the nanoemulsion can be passed though the homogenizer from about two to about five times. The particle size depends inversely on both the number of passages and on the operating pressure. See FIG. 5.

In addition to the above described methods, one can produce a small particle size nanoemulsion directly, without premixing. The direct use of, for example, either a microfluidizer or a high pressure homogenizer, as described above, can result in a small particle size nanoemulsion with the properties discussed above for a small particle size nanoemulsion produced from a premixed standard nanoemulsion.

Small particle size nanoemulsions can have a consistency ranging from a semi-solid cream to a watery liquid similar to skim milk. Creamy emulsions can be used as-is or mixed with water.

A nanoemulsion can be prepared in a diluted or an undiluted form. In one embodiment a nanoemulsion shows suitable stability in both diluted and undiluted forms. By suitable stability, it is meant that the emulsions do not show any signs of separation (oil phase from aqueous phase) for at least 6 months. In another embodiment a nanoemulsion does not show any sign of separation up to about 2 years. In a further embodiment, a nanoemulsion does not show any sign of separation for up to about 3 years. Settling of the diluted emulsions is an acceptable characteristic and does not indicate separation of an oil phase from an aqueous phase. Settling is due to separation of emulsions from its diluent, not an oil phase separating from an aqueous phase. Such settling is readily reversed by simple shaking of the nanoemulsion, while separation of the concentrated emulsions are not reversed by simple mixing, requiring instead re-emulsification.

The emulsions can also contain a first nanoemulsion emulsified within a second nanoemulsion, wherein the first and second emulsions can each contain an aqueous phase, an oil phase, and a surfactant. Either one or both nanoemulsions of this composition can contain an anti-inflammatory agent. The oil phase of each of the first and second nanoemulsion can contain an oil and an organic solvent. The first and second nanoemulsion can be the same or different. A nanoemulsion can also contain a first nanoemulsion re-emulsified to form a second nanoemulsion.

One useful parameter for characterizing a nanoemulsion is “zeta potential.” Zeta potential is the electrical potential of a shear plane (an imaginary surface separating a thin layer of liquid that shows elastic behavior) bound to a solid surface that shows normal viscous behavior The stability of hydrophobic colloids depends, in part, on the zeta potential. Zeta potential of a nanoemulsion can be about −50 mV to about +50. In one embodiment, the zeta potential of the emulsions can be greater than or equal to about +10 mV. In another embodiment, the zeta potential is greater than or equal to about +20 mV. In a further embodiment, the zeta potential of the emulsions can be less than or equal to about +45 mV, less than or equal to about +40 mV or less than or equal to about +30 mV.

In one embodiment a nanoemulsion composition having anti-inflammatory activity, comprising optional therapeutic agents, can be provided in the form of pharmaceutically acceptable compositions. The terms “pharmaceutically acceptable” or “pharmacologically acceptable” refer to compositions that do not produce significant adverse, allergic, or other untoward reactions when administered to an animal or a human

Compositions for pharmaceutical use typically comprise a pharmaceutically acceptable carrier, for example, solvents, dispersion media, coatings, isotonic and absorption delaying agents and the like, and combinations comprising one or more of the foregoing carriers as described, for instance, in Remington's Pharmaceutical Sciences, 15th Ed. Easton: Mack Publishing Co. pp. 1405-1412 and 1461-1487 (1975), and The National Formulary XIV 14th Ed., Washington: American Pharmaceutical Association (1975). Suitable carriers include, but are not limited to, calcium carbonate, carboxymethylcellulose, cellulose, citric acid, dextrate, dextrose, ethyl alcohol, glucose, hydroxymethylcellulose, lactose, magnesium stearate, maltodextrin, mannitol, microcrystalline cellulose, oleate, polyethylene glycols, potassium diphosphate, potassium phosphate, saccharose, sodium diphosphate, sodium phosphate, sorbitol, starch, stearic acid and its salts, sucrose, talc, vegetable oils, water, and combinations comprising one or more of the foregoing carriers. The use of such media and agents for pharmaceutically active substances is well known in the art. Except insofar as any conventional media or agent is incompatible with the emulsions of the present invention, their use in therapeutic compositions is contemplated. Supplementary active ingredients also can be incorporated into the compositions.

For topical applications, pharmaceutically acceptable carriers can take the form of a liquid, cream, foam, lotion, or gel, and may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly used in pharmaceutical compositions for topical administration.

C. Methods of Using Nanoemulsion Compositions to Inactivate a Pathogenic Microorganism

Nanoemulsion compositions having anti-inflammatory activity are particularly useful in applications where inactivation of pathogenic microorganisms is desired and where an anti-inflammatory is beneficial. The term inactivating means killing, eliminating, neutralizing, or reducing the capacity of a pathogenic microorganism to infect a host on contact. Nanoemulsion compositions are useful for decreasing the infectivity, morbidity, and/or rate of mortality associated with a variety of pathogenic microorganisms.

A method of inactivating a pathogenic microorganism comprises contacting the pathogenic microorganism with an amount a nanoemulsion composition that is effective to inactivate the microorganism. The step of contacting can involve contacting any substrate which may be or is suspected to be contaminated with a nanoemulsion composition. By substrate it is meant, without limitation any subject, such as a human or an animal, and contact can be in vivo or ex vivo. A pathogenic microorganism can be, without limitation, a bacteria, a virus, a fungus, a protozoan or a combination thereof.

The step of contacting can be performed for any amount of time sufficient to inactivate a microorganism or deliver the anti-inflammatory agent. In one embodiment, inactivation occurs within about 5 minutes to about 10 minutes after initial contact. However, it is understood that when the emulsions are used in a therapeutic context and applied topically or systemically, the inactivation may occur over a longer period of time, for example, 5, 10, 15, 20, 25 30, 60 minutes or longer after administration.

The step of contacting can be performed using any appropriate means of application. For example, compositions can be administered by spraying, fogging, misting, exposure to aerosols, wiping with a wet or saturated cloth or towlette, drenching, immersing.

Nanoemulsion compositions can be used to inactivate vegetative bacteria and bacterial spores upon contact. Bacteria inactivated by nanoemulsion compositions can be Gram negative or Gram positive bacteria. Gram negative bacteria include, for example and without limitation, Vibrio, Salmonella, Shigella, Pseudomonas, Escherichia, Klebsiella, Proteus, Enterobacter, Serratia, Moraxella, Legionella, Bordetella, Gardnerella, Haemophilus, Neisseria, Brucella, Yersinia, Pasteurella, Bacteroids, and Helicobacter. Gram positive bacteria include, for example, and without limitation, Bacillus, Clostridium, Arthrobacter, Micrococcus, Staphylococcus, Streptococcus, Listeria, Corynebacteria, Planococcus, Mycobacterium, Nocardia, Rhodococcus, and acid fast Bacilli such as Mycobacterium. In one embodiment, nanoemulsion compositions can be used to inactivate Bacillus, including, without limitation B. anthracis, B. cereus, B. circulans, B. subtilis, and B. megaterium. Nanoemulsion compositions can also be used to inactivate Clostridium, e.g., C. botulinum, C. perfringens, and C. tetani. Other bacteria that can be inactivated by a nanoemulsion include, but are not limited to, H. influenzae, N. gonorrhoeae, S. agalactiae, S. pneumonia, S. pyogenesand V. cholerae (classical and Eltor), and Yersinia, including, Y. pestis, Y. enterocolitica, and Y. pseudotuberculosis. In another embodiment, the bacteria is B. anthracis. In another embodiment, the bacteria is Mycobaterium tuberculosis.

Contacting a bacterial spore with a nanoemulsion inactivates the spore. Without being bound to any theory, it is proposed that the sporicidal ability of the nanoemulsions is by initiation of germination without complete reversion to the vegetative form, leaving the spore susceptible to disruption by the emulsions. Induction of germination using germination enhancers such as inosine and L-alanine can result in acceleration of the sporicidal activity of the nanoemulsion, while inhibition of initiation of germination with D-alanine can delay sporicidal activity. This unique action of a nanoemulsion, which can be better in efficiency than 1% bleach, is interesting because Bacillus spores are generally resistant to most disinfectants including many commonly used detergents. The sporicidal effect can start almost immediately. In one embodiment the sporicidal effect occurs within 30 minutes of contact with a nanoemulsion.

Contacting a nanoemulsion composition with a virus can inactivate a virus. The effect of nanoemulsion compositions on viral agents can be monitored using any suitable means, such as, for example, plaque reduction assay (PRA), cellular enzyme-linked immunosorbent assay (ELISA), P-galactosidase assay, and electron microscopy (EM). Viruses which can be inactivated by contact with a nanoemulsion composition include, without limitation, and virus of the families Baculoviridae, Herpesviridae, Iridoviridae, Poxyiridae, “African Swine Fever Viruses,” Adenoviridae, Caulimoviridae, Myoviridae, Phycodnaviridae, Tectiviridae, Papovaviridae, Circoviridae, Parvoviridae, Hepadnaviridae, Cystoviridae, Birnaviridae, Reoviridae, Coronaviridae, Flaviviridae, Togaviridae, “Arterivirus,” Astroviridae, Caliciviridae, Picornaviridae, Potyviridae, Retroviridae, Orthomyxoviridae, Filoviridae, Paramyxoviridae, Rhabdoviridae, Arenaviridae, and Bunyaviridae. In one embodiment, the virus is herpes, pox, papilloma, corona, influenza, hepatitis, sendai, sindbis and vaccinia viruses, west nile, hanta, and viruses which cause the common cold.

In yet another embodiment, contacting a nanoemulsion with a fungus inactivates the fungus. In one embodiment, the fungus is a yeast, such as, for example various species of Candida (e.g., Candida albicans) or filamentous yeast including but not limited to Aspergillus species or dermatophytes such as Trichophyton rubrum, Trichophyton mentagrophytes, Microsporum canis, Microsporum gypseum, and Epiderophyton floccosum, and types thereof, as well as others.

The methods and compositions, or components of the methods and compositions can be formulated in a single formulation, or can be separated into binary formulations for later mixing during use, as may be desired for a particular application. Such components can advantageously be placed in kits for use against microbial infections, decontaminating instruments and the like. Such kits may contain all of the essential materials and reagents required for the delivery of the formulations to the site of their intended action as well as any desired instructions.

For in vivo use, the methods and compositions may be formulated into a single or separate pharmaceutically acceptable syringeable composition. In this case, the container means may itself be an inhalant, syringe, pipette, eye dropper, or other like apparatus, from which the formulation may be applied to an infected area of the body, such as the lungs, injected into an animal, or even applied to and mixed with the other components of the kit.

A kit also can include a means for containing the vials in close confinement for commercial sale (e.g., injection or blow-molded plastic containers into which the desired vials are retained). Irrespective of the number or type of containers, the kits also may comprise, or be packaged with, an instrument for assisting with the injection/administration or placement of the ultimate complex composition within the body of an animal. Such an instrument may be an inhalant, syringe, pipette, forceps, measured spoon, eyedropper, or any such medically approved delivery vehicle.

Actual amounts of nanoemulsions and additives in the compositions can be varied so as to provide amounts effective to inactivate vegetative as well as sporular microorganisms and pathogens. Accordingly, the selected amounts will depend on the nature and site for treatment, the desired response, the desired duration of biocidal action, the condition of the subject being treated, and other factors. A nanoemulsion composition can comprise, for example, about 0.001% to about 100% nanoemulsion per milliliter of liquid composition. In one embodiment, a nanoemulsion composition can contain about 0.01% to about 90% nanoemulsion per milliliter of liquid. These are merely exemplary ranges. A nanoemulsion composition can also comprise greater than about 0.25%, about 1.0%, about 5%, about 10%, about 20%, about 35%, about 50%, about 65%, about 80%, about 90%, or about 95% of nanoemulsion per milliliter of liquid composition.

The small particle size nanoemulsions as described herein are more stable than standard emulsions under a variety of conditions, showing substantially no observable separation or settling for up to one month, preferably up to four months, more preferably up to or more than one year, up to about 21° C., preferably up to 40° C. Such stability is at no dilution, up to 2.5% dilution, up to 10% dilution, more preferably up to 50% dilution or higher.

The small particle size nanoemulsions perform equal to or better than standard emulsions in inactivating a pathogenic microorganism, exhibiting a less than 10% failure rate, preferably a less than 5% failure rate, more preferably a less than 1% failure rate, and most preferably a 0% failure rate against pathogens. The invention is further illustrated by the following non-limiting examples.

1. Prevention and Treatment of Infection

Nanoemulsion compositions having anti-inflammatory activity are useful for the prevention and treatment of infection. A method of inactivating a pathogenic microorganism comprises contacting a subject infected with or suspected to be infected with the microorganism with a nanoemulsion composition comprising an aqueous phase, an oil phase, one or more anti-inflammatory agents and one or more surfactants. The oil phase comprises an oil and an organic solvent, as discussed above. The nanoemulsion particles have an average diameter of less than or equal to about 250 nm. In one embodiment, the particles have an average diameter of less than or equal to about 200 nm, less than or equal to about 150 nm, less than or equal to about 100 nm, or less than or equal to about 50 nm.

The pathogenic microorganism may have systemically infected the subject or on the surface of the subject. Where the microorganism is not on the subject, the nanoemulsion composition is delivered to the site of infection by any suitable method, for example injection, oral administration, suppositories, and the like. In one embodiment the subject is an animal. In a further embodiment, the animal is a human.

Exemplary infected states that can be treated or prevented with nanoemulsions include, but are not limited to, bacterial, fungal, protozoal, and/or viral vaginal infection, sexually transmitted diseases (STDs), skin infections such as, acne, impetigo, athlete's foot, onychomycosis, candidiasis and other acute fungal infections, herpes simplex and zoster and infections associated with psoriasis or other skin inflammatory diseases. In one embodiment, an infected state is particularly susceptible to topical treatment. As used herein, “infected states” is inclusive of contamination with pathogenic microorganisms, and treatment and prevention of such infected states includes, but is not limited to, wound decontamination, decontamination of skin, airways, and/or mucosal surfaces (e.g., with anthrax spores, viruses, bacteria, and/or fungi); and the like. Nanoemulsion compositions can also be used as a surgical irrigant. The emulsions can be used in the personal health care industry in deodorants, soaps, body wash, acne/dermatophyte treatment agents, treatments for halitosis, and skin disinfecting.

Nanoemulsion compositions can be used in a variety of combination therapies, particularly those directed to microorganisms. This approach is often advantageous in avoiding the problems encountered as a result of multidrug resistance, for example.

In one embodiment, a nanoemulsion composition having anti-inflammatory activity can be used in the prevention or treatment of genital infections. Such sexually transmitted genital infections include, but are not limited to genital herpes, human papilloma virus (HPV), human immunodeficiency virus (HIV), trichomoniasis, gonorrhea, syphilis, and chlamydia. A nanoemulsion composition having anti-inflammatory activity can be applied to the genitals either before or after sexual intercourse or both before and after sexual intercourse. In one embodiment, a nanoemulsion composition is introduced into the vagina of a female, at about the time of sexual. In another embodiment, a nanoemulsion composition is introduced into the vagina of a female prior to intercourse. A nanoemulsion composition can also be administered to other mucous membranes. Application of a nanoemulsion composition to genitalia can be accomplished using any appropriate means including, for example, ointments, jellies, inserts (suppositories, sponges, and the like), foams, and douches.

A nanoemulsion composition having anti-inflammatory activity can also be used in the treatment of nonsexually transmitted genital infections, such as fungal, protozoan, bacterial infections. Fungal infections treatable with a nanoemulsion composition include, but are not limited, to tinea, candida (e.g., Candida albicans). Nonsexually treated bacterial infections treatable with a nanoemulation include, but are not limited, nonspecific vaginitis and bacterial vaginitis caused by, for example, Gardnerella vaginalis, Gardneralla mobiluncus, and Mycoplasma hominis.

Nanoemulsion composition having anti-inflammatory activity can also be used for the prevention and treatment of respiratory infection. Nanoemulsion compositions can be used to prevent infection by, without limitation, the common cold, influenza, tuberculosis, legionnaire's disease, and acute respiratory syndrome (SARS). In one embodiment, a nanoemulsion composition is applied to the respiratory passages using, for example, a nasal spray, such that the spray coats the respiratory passages before exposure to these pathogens. In another embodiment, this use can substantially inactivate or eliminate a respiratory pathogen preventing the pathogen from inducing a pathogenic response. The use of a nanoemulsion in the prevention and treatment of a respiratory infection can also stimulate an immunological response against a specific pathogen which can protect from further exposure to the same pathogen.

### EXAMPLE 1

### Comparison of Standard Emulsions and Small Particle Size Nanoemulsions

The nanoemulsions are described by the components of the nanoemulsion according to Table 1. Unless otherwise noted, the oil is soybean oil. In the formulations, the detergent is listed first, followed by the volume percentage of the detergent (e.g., W₂₀5 refers to 5 vol % of Tween 20). In the formulations, the designation L2 refers to a small particle size nanoemulsion produced by a microfluidizer, while the absence of the L2 designation refers to a standard nanoemulsion (i.e., average particle sizes of 250 nm to about 1 micrometer). The designation L3 refers to nanoemulsions produced using an Avesting high pressure homogenizer.

TABLE 1

| Component | Symbol |
|---|---|
| Tween 20 | W₂₀ |
| Ethanol | E |
| Cetylpyridinium chloride | C |
| EDTA | ED |
| Triton X-100 | X |
| Tributyl phosphate | P |
| Glycerol | G |
| Benzalkonium chloride | BA |

A first nanoemulsion is produced from a mixture containing 548 milliliters of water, 2.24 grams of EDTA, 25 grams of cetylpyridiunium chloride, 125 milliliters of Tween 20, 200 milliliters of ethanol and 1600 milliliters of soybean oil. The first nanoemulsion is pre-mixed with a Silverson L4RT mixer and a fine emulsifier screen for 10 minutes at 10,000±500 revolutions per minute.

The first nanoemulsion is then processed in a Microfluidics M-110EH microfluidizer processor using an H210Z (200 μm) chamber downstream of an H230Z (400 μm) chamber. The first nanoemulsion is passed through the microfluidizer 3 to 4 times at a pressure of 3,500±500 pounds per square inch (psi) using cooling ice in the tray surrounding the chambers. The small particle size nanoemulsion produced is referred to as W₂₀EC ED L2.

The second nanoemulsion is then diluted with distilled water to produce a series of diluted nanoemulsions. The water and the nanoemulsion can be mixed by shaking, for example, until the nanoemulsion is incorporated into the water. Exemplary diluted nanoemulsions are as shown in Table 2. The percentage shown refers to the volume percentage of the nanoemulsion in the dilution.

TABLE 2

| Formulation | water | W₂₀5EC ED L2 |
|---|---|---|
| 50% W₂₀5EC ED L2 | 500 mL | 500 mL |
| 20% W₂₀5EC ED L2 | 800 mL | 200 mL |
| 10% W₂₀5EC ED L2 | 900 mL | 100 mL |
| 5% W₂₀5EC ED L2 | 950 mL | 50 mL |
| 2.5% W₂₀5EC ED L2 | 975 mL | 25 mL |

### EXAMPLE 2

### Method of Making a Small Particle Size Nanoemulsion

A standard nanoemulsion (i.e., particles sizes of 250 nm to 5 micrometers) is formed as follows. A mixture of 22 vol % distilled water, 1 wt/vol % cetylpyridinium chloride, 5 vol % Tween 20, 64 vol % soybean oil, and 8 vol % ethanol based on the total volume of the mixture is formed. The nanoemulsion is formed by mixing for 5 minutes at 10,000±500 revolutions per minute with a Silverson L4RT mixer with a standard mixing assembly and a fine emulsion screen. The standard nanoemulsion is denoted as W₂₀5EC.

A small particle size nanoemulsion is formed by passing the W₂₀5EC nanoemulsion 4 times through a Microfluidics M-110EH microfluidizer processor using an H210Z (200 μm) chamber downstream of an H230Z (400 μm) chamber. The small particle size nanoemulsion is denoted as W₂₀5EC L2.

After formation, the W₂₀5EC and W₂₀5EC L2 emulsions are diluted with water for further testing. Particle sizes are determined by Particle Sizing Systems (PSS) Nicomp Model 380. The samples are diluted 1/2000 in distilled water to measure the particle size. The formulations and data are shown in Table 3.

TABLE 3

| | | Amount of | | | | Average |
|---|---|---|---|---|---|---|
| Formulation | | nano- | | Amount | | Particle Size, |
| No. | Formulation | emulsion | | of water | | nm |
| 1 | W₂₀5EC | — | | — | | 421.4 |
| 2 | 50% W₂₀5EC | 90 | mL | 90 | mL | 454 |
| 3 | 20% W₂₀5EC | 36 | mL | 144 | mL | 437.5 |
| 4 | 10% W₂₀5EC | 18 | mL | 162 | mL | 418.8 |
| 5 | 5% W₂₀5EC | 9 | mL | 171 | mL | 427.4 |
| 6 | 2.5% W₂₀5EC | 4.5 | mL | 175.5 | mL | 470.3 |
| 7 | W₂₀5EC L2 | — | | — | | 152 |
| 8 | 50% W₂₀5EC L2 | 90 | mL | 90 | mL | 99.3, 219.5* |
| 9 | 20% W₂₀5EC L2 | 36 | mL | 144 | mL | 144.2 |
| 10 | 10% W₂₀5EC L2 | 18 | mL | 162 | mL | 153 |
| 11 | 5% W₂₀5EC L2 | 9 | mL | 171 | mL | 177.8 |
| 12 | 2.5% W₂₀5EC L2 | 4.5 | mL | 175.5 | mL | 157.7 |

*When there is wide range of particle sizes (Nicomp reading), two methods of calculation are used

As shown in Table 3, dilution of the emulsions does not appreciably affect the particle size of either the standard nanoemulsion or the small particle size nanoemulsion. The average particle size for the W₂₀5EC emulsions is about 400 to about 500 nm (samples 1-6) and for the W₂₀5EC L2 emulsions is about 140 to about 220 nm (samples 7-12).

### EXAMPLE 3

### Effect of Microfluidizer Chamber Size on the Size of Small Particle Size Nanoemulsion Particles

A W₂₀5G BA2 nanoemulsion is passed through different combinations of microfluidizer chambers as shown in Table 4. The W₂₀5G BA2 L2 small particle size nanoemulsion is made with 1 pass with a Silverson L4RT mixer and 4 passes through a microfluidizer. Combinations of chamber having 75, 200, 400 micrometer microchannels are used to determine the relationship between the size of the microchannels and the size of the particles produced.

TABLE 4

| | First chamber, | Second chamber, | |
|---|---|---|---|
| Sample | μm | μm | Particle size, nm |
| 1 | 75 | 100 | 174 |
| 2 | 100 | 75 | 165 |
| 3 | 75 | 200 | 185 |
| 4 | 200 | 75 | 180 |
| 5 | 75 | 400 | 211 |
| 6 | 400 | 75 | 199 |

As shown in Table 4, the chamber size utilized in the microfluidizer, when varied between 75 and 400 μm, does not significantly affect the particle size of the emulsions. In all cases, the particle size is less than or equal to about 250 nm.

### EXAMPLE 4

### Effect of Number of Passes Through the Microfluidizer on Emulsion Particle Size

A W₂₀5G BA2 nanoemulsion is formed using either a Silverson L4RT mixer (high shear) or a household hand mixer (low shear). The nanoemulsion is then passed through the microfluidizer for 1 to 6 passes and the particle size measured. The relationship between the number of passes in the microfluidizer and the particle size of the emulsions are shown in Table 5 and FIG. 5.

TABLE 5

| | | Number of | Nanoemulsion Particle Size (nm) |
|---|---|---|---|
| | Type of First | Passes Through | (three independent experiments |
| Sample | Mixer | Microfluidizer | with different emulsion lots) |
| 1 | High shear | 1 | 183, 221, 267 |
| 2 | High shear | 2 | 183, 205, 195 |
| 3 | High shear | 3 | 210, 202, 201 |
| 4 | High shear | 4 | 155, 156, 156 |
| 5 | High shear | 4 | 220, 157, 180 |
| 6 | High shear | 5 | 157, 132, 158 |
| 7 | High shear | 6 | 196, 161, 168 |
| 8 | Low shear | 0 | 426, 529, 522 |
| 9 | Low shear | 1 | 275, 210, 205 |
| 10 | Low shear | 2 | 218, 168, 218 |
| 11 | Low shear | 3 | 183, 151, 129 |
| 12 | Low shear | 4 | 182, 179, 180 |

As shown in Table 5 and FIG. 5, the number of passes through the microfluidizer does not have a large effect on the nanoemulsion particle size. As shown in Sample 4 and 5, 4 passes through the microfluidizer produces particle sizes consistently below 250 nm. Regarding high shear versus low shear mixing of the starting emulsion, while high shear mixing can produce a more consistent particle size distribution than the low shear mixing, high shear mixing of the starting emulsion is not required to produce the small particle size nanoemulsions.

### EXAMPLE 5

### Combined Effects of Number of Passes through the Microfluidizer and Microfluidizer Chamber Size

The effect of both the number of passes through the microfluidizer and the chamber size in the microfluidizer are studied for different formulations. The starting emulsions are prepared using either a Silverson L4RT mixer (“Silv”) or a Ross HSM-410× high shear mixer with a 3 inch X-series rotor/stator pre-set to a 0.010 gap (Ross) in order to determine the effect of mixing method on the particle size of the starting nanoemulsion (i.e., prior to passage through the microfluidizer). The L2 emulsions are produced by passing a standard nanoemulsion produced by Silverson mixing through a microfluidizer. The particle sizes are shown in Table 6.

TABLE 6

| | | | Interactive | Number | |
|---|---|---|---|---|---|
| | | High shear | chamber | of | Particle size, |
| Sample | Formulation | Mixer type | used | passages | nm |
| 1 | Nanowash + alcohol* | Silv | — | — | 410-486 |
| 2 | W₂₀5G BA2 | Silv, 5 | — | — | 304-371 |
| | | minutes | | | |
| | | mixing | | | |
| 3 | W₂₀5G BA2 | Silv, 20 min | — | — | 283-340 |
| | | mixing | | | |
| 4 | S8G | Silv | — | — | 350 |
| 5 | W₂₀5EC | Silv | — | — | 381 |
| 6 | W₂₀5G | Silv | — | — | 486 |
| 7 | W₂₀5G BA2 | Ross | — | 1 | 260 |
| 8 | W₂₀5G BA2 | Ross | | 2 | 247 |
| 9 | W₂₀5G BA2 | Ross | | 3 | 281 |
| 10 | W₂₀5G BA2 | Ross | | 4 | 229-254 |
| 11 | W₂₀5G BA2 | Microfluidizer | 400, 200 | 2 | 196 |
| 12 | W₂₀5G BA2 | Microfluidizer | 400, 200 | 3 | 195 |
| 13 | W₂₀5G BA2 | Microfluidizer | 200, 200 | 3 | 173 |
| 14 | W₂₀5G BA2 | Microfluidizer | 75, 200 | 3 | 210 |
| 15 | W₂₀5G BA2 | Microfluidizer | 75, 200 | 3 | 235 |
| 16 | W₂₀5G BA2 | Microfluidizer | 200, 400 | 3 | 179 |
| | | | then | | |
| | | | diluted | | |
| | | | using 75, | | |
| | | | 200 | | |
| 17 | S8G** | Microfluidizer | 75, 200 | 3 | 161 |
| 18 | W₂₀5EC | Microfluidizer | 75, 200 | 3 | 178 |
| 19 | W₂₀5EC | Microfluidizer | 75, 200 | 3 | 158 |
| 20 | W₂₀5G | Microfluidizer | 75, 200 | 3 | 223 |
| 21 | W₂₀5GC*** | Microfluidizer | 400, 200 | 3 | 189, 200, |
| | | | | | 225, 226 |
| 22 | X₈GC | Microfluidizer | 400, 200 | 3 | 130, 145 |
| 23 | X₈E₆G₂**** | Microfluidizer | 400, 200 | 3 | 249 |

*1% W₂₀5 GBA2 + 2 mM EDTA + 20% ethanol
**8% SDS, 6% glycerol, 64% soybean oil, 20% water
***5% Tween 20, 8% glycerol, 1% cetylpyridinium chloride, 64% soybean oil, 22% water
****8% Triton X100, 6% ethanol, 2% glycerol, 64% soybean oil, 20% water

As shown in Table 6, the Silverson high shear mixer (samples 1-6) produces particle sizes of about 300 nm to about 500 nm. The Ross high shear mixer (Samples 7-10) produces particle sizes of 260 nm after 1 pass to about 229 to 254 nm after 4 passes. The Ross high shear mixer is thus capable of producing smaller particle sizes than the Silverson mixer. Also shown in Table 6 is that the samples passed through the microfluidizer (samples 11-23) have smaller particle sizes than the samples mixed with either high shear mixer (samples 1-10).

Regarding the samples passed through the microfluidizer, as shown in samples 11 and 12, similar particle sizes are obtained with either 2 or 3 passes through the microfluidizer. Samples 13-16 show that changing the microchannel size of the microfluidizer chamber does not decrease the particle size of the emulsions. Samples 17-23 illustrate that, independent of the formulation of the emulsions, emulsions having particle sizes of less than about 250 nm can be formed by passing the emulsions through a microfluidizer.

### EXAMPLE 6

### Particle Sizes and Zeta Potentials for Different Nanoemulsion Formulation

In this experiment, the particle sizes and zeta potentials for different small particle size nanoemulsion formulations are determined. The emulsions are formed by passing a starting nanoemulsion through the microfluidizer for 3 passes using the H230Z+H210Z chambers. The particle size and zeta potential are measured by Nicomp 380 Particle sizer. The data are shown in Table 7.

TABLE 7

| | | | Zeta |
|---|---|---|---|
| Sample | Formulation | Particle Size | (mV) |
| 1 | 1% W₂₀5G BA2 L2 + 2 mM EDTA | 186 | 11 |
| 2 | W₂₀5G BA2 L2 in water | 183 | 27 |
| 3 | W₂₀5GC L2 | 168-236 | 30-33 |
| 4 | W₂₀5G SA2 OA2 L2* | 226 | 33 |
| 5 | W₂₀5E SA3 L2 | 154 | 31 |
| 6 | W₂₀5E SA3 L2 + 2 mM EDTA | 131 | 12 |
| 7 | W₂₀5G SA3 L2** | 215 | 32 |
| 8 | W₂₀5G SA3 L2 + 2 mM EDTA | 187, 191 | 12 |
| 9 | W₂₀5E L2 | 189 | −25 |
| 10 | W₂₀5EC L2, premixed | 156, 182 | 31 |
| 11 | W₂₀5EC L2 | 146 | 41 |

*5% Tween 20, 8% glycerol, 2% sterylamine, 2% oleyl alcohol, 61% soybean oil, 21% water
**5% Tween 20, 8% glycerol, 3% Sterylamine, 61% Soybean oil, 23% water

As shown in Table 7, all of the formulations have particle sizes of less than or equal to about 250 nm.

### EXAMPLE 7

### Stability of Nanoemulsions

A W₂₀5EC nanoemulsion was formed containing 5% Tween-20, 8% ethanol, 1% cetylpyridinium chloride, 64% soybean oil, and the balance water. A W₂₀5EC L2 nanoemulsion is formed using 2 passes on a microfluidizer. A W₂₀5GC nanoemulsion is formed containing 5% Tween-20, 8% glycerol, 1% cetylpyridinium chloride, 64% soybean oil, and the balance water. A W₂₀5GC L2 nanoemulsion is formed using 2 passes on a microfluidizer. An X8P nanoemulsion is formed using 8% Triton X-100, 8% tributyl phosphate, and the balance water.

Stability is determined by evaluating the physical appearance of the emulsions. As used herein, creaming is the presence of a white layer of creamy material on top of the nanoemulsion that is more opaque than the rest of the nanoemulsion. Settling is a gradual decrease in opacity of the nanoemulsion from top to bottom due to separation of the more dense diluent (water) at the bottom from the less dense nanoemulsion at the top. The water appears as transparent layer at the bottom of the vial. Settling is classified as follows: Mild settling: the nanoemulsion appears cloudy with a gradient of “cloudiness” where it gets more opaque as you go upwards. Moderate settling: a partially clear aqueous solution appears on the bottom of the sample. The rest of the nanoemulsion appears cloudy with a gradient of cloudiness getting more opaque as you go up. Some creaming may be on the surface. Severe settling: nanoemulsion has the appearance of three distinct layers, a partially clear bottom, cloudy middle, and creamy top. Extreme settling: only two layers, a thick partially clear bottom and a thin creamy top.

Separation is the phase separation of the nanoemulsion ingredients. Separation is classified as follows: Mild separation: the surface of the nanoemulsion shows few visible oil droplets. Moderate separation: the surface of the nanoemulsion has a film of oil. The bottom of the nanoemulsion may have a clear aqueous layer. Severe separation: nanoemulsion has the appearance of three distinct layers, a clear aqueous layer on the bottom, a white or cloudy middle layer and a dense oily layer on the top. Extreme separation: total separation into an oil layer on top and water on bottom.

The ambient storage stability test includes storing the neat emulsions in polypropylene bottles or centrifuge tubes at room temperature (22-25° C.). Containers may be mixed or opened during the observation period. The emulsions are observed for separation or any other changes in appearance. The observation period is varied due to different manufacturing dates of the emulsions. The data for W₂₀5EC emulsions are shown in Table 8.

TABLE 8

| | Days in | Bottle | Type of | |
|---|---|---|---|---|
| Sample | storage | fullness | container | Appearance |
| 1 | 579 | ¼ | 125 ml PP | severe separation 93%: |
| | | | | <7% nanoemulsion |
| | | | | between oil & water |
| 2 | 619 | ¼ | 125 ml PP | extreme separation |
| 3 | 505 | ⅔ | 250 ml PP | moderate separation- |
| | | | | 6% oil |
| 4 | 585 | ⅔ | 250 ml PP | moderate separation- |
| | | | | 8% oil |
| 5 | 457 | ⅔ | 250 ml PP | mild separation- 1% oil |
| 6 | 497 | ⅔ | 250 ml PP | moderate separation- |
| | | | | 1.5% oil |
| 7 | 184 | full | 125 ml PP | mild-oil drop in air space |
| 8 | 224 | full | 125 ml PP | mild-oil drop in air space |
| 9 | 184 | ¾ | 125 ml PP | mild separation- 1% oil |
| | | | | film |
| 10 | 224 | ¾ | 125 ml PP | moderate separation- |
| | | | | 2% oil film |
| 11 | 184 | ⅔ | 125 ml PP | mild separation- 4% oil |
| 12 | 224 | ⅔ | 125 ml PP | moderate separation- |
| | | | | 6% oil |
| 13 | 112 | ¼ | 500 ml PP | intact |
| 14 | 152 | ¼ | 500 ml PP | moderate separation- |
| | | | | 3% oil |
| 15 | 33 | full | 30 ml PP | intact |
| 16 | 74 | full | 30 ml PP | mild separation- 1 of 4 |
| | | | | vials with oil film |
| 17 | 74 | ½ | 250 ml PP | mild separation |

*PP = polypropylene

The data for W₂₀5EC L2 emulsions are shown in Table 9.

TABLE 9

| | Days in | Bottle | Type of | |
|---|---|---|---|---|
| Sample | storage | fullness | container | Appearance |
| 18 | 116 | full | 30 ml PP | intact |
| 19 | 157 | full | 30 ml PP | intact |
| 20 | 74 | ¼ | 60 ml PP | intact |
| 21 | 115 | ¼ | 60 ml PP | intact |
| 22 | 75 | full | 500 ml PP | intact |
| 23 | 115 | full | 500 ml PP | intact |
| 24 | 33 | full | 30 ml PP | intact |
| 25 | 74 | full | 30 ml PP | intact |

As shown in Tables 8 and 9, the small particle size nanoemulsions are more stable at room temperature than comparable standard emulsions. Batches of standard W₂₀5EC neat nanoemulsion stored at ambient temperatures longer than 5 months show oil forming a film or layer on the surface of the nanoemulsion. The thickness of the oil layer is variable and may be related in part to the amount of air in the storage container in addition to the number of times the container has been entered.

Batches of smaller particle size W₂₀5EC L2 neat nanoemulsion are stored at ambient temperatures for up to 4 months. No settling or separation is observed in these batches.

Accelerated stability testing is also performed as follows. Glass vials are filled with 20 milliliters of neat, 10% diluted and 2.5% diluted nanoemulsion. The emulsions are stored at 55° C. and observed 3 times a week for changes in physical appearance. One additional set of vials for the W₂₀5EC L2 emulsions is filled completely (about 25 milliliters) to eliminate air during storage. These full vials are inverted at day 7 to facilitate observation of creaming and separation.

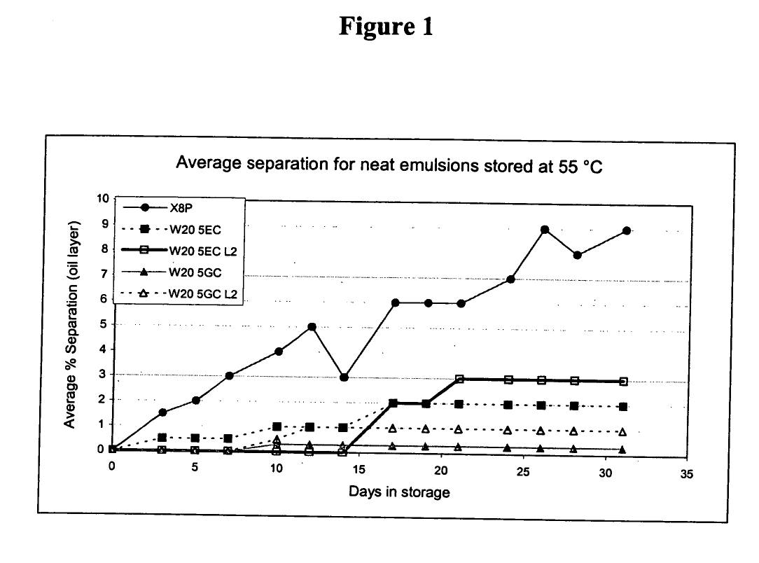

Neat emulsions (100%) of standard W₂₀5EC and small particle size nanoemulsion W₂₀5EC L2 under accelerated stability testing at 55° C. show a film of oil separating after 4 and 5 days, respectively (FIG. 1 and Table 10).

TABLE 10

| | Average Days | | | Average Days | | |
|---|---|---|---|---|---|---|
| | to Mild or | | | to Severe or | | |
| | Moderate Separation | | | Extreme Separation | | |
| Nanoemulsion | Neat | 10% | 2.50% | Neat | 10% | 2.50% |
| X8P | 3 | N | N | 10 | N | N |
| W₂₀5EC | 4.3 | N | N | N | N | N |
| W₂₀5EC L2 | 5.3 | N | N | N | N | N |
| W₂₀5EC L2 full* | N | N | N | N | N | N |
| W₂₀5GC | 5.7 | N | N | N | N | N |
| W₂₀5GC L2 | 8.7 | N | N | N | N | N |

N = No separation

For comparison, the X8P neat nanoemulsion shows signs of instability with a distinct clear aqueous layer on the bottom and a 5% oil layer on the surface. Neat emulsions of both W₂₀5GC and W₂₀5GC L2 show yellowing of the oil film on the surface of the nanoemulsion, whereas for W₂₀5EC and W₂₀5EC L2, the oil film is colorless. The neat small particle size nanoemulsions are stable for 1-3 days longer than the standard emulsions.

No diluted nanoemulsion (10% or 2.5%) shows separation of oil after 4 weeks observation at 55° C. (Table 10).

Table 11 shows the settling observed for the nanoemulsions after accelerated aging.

TABLE 11

| | Average Days | | | Average Days | | |
|---|---|---|---|---|---|---|
| | to Mild or | | | to Severe or | | |
| | Moderate Settling | | | Extreme Settling | | |
| Nanoemulsion | Neat | 10% | 2.50% | Neat | 10% | 2.50% |
| X8P | N | 3 | 3 | N | 10 | 10 |
| W₂₀5EC | N | 3 | 3 | N | 19 | 10 |
| W₂₀5EC L2 | N | 10.6 | 5 | N | N | N |
| W₂₀5EC L2 full* | N | N | 5 | N | N | N |
| W₂₀5GC | N | 5 | 3 | N | 26 | 19 |
| W₂₀5GC L2 | N | 10 | 3 | N | N | N |

On average, the small particle size nanoemulsions exhibit less oil separation and less separation of the oil and water layers than the standard emulsions (Table 10). The small particle size nanoemulsions exhibit comparable settling and creaming to the standard emulsions when undiluted and improved stability when diluted to 10% or 2.5% (Table 11).

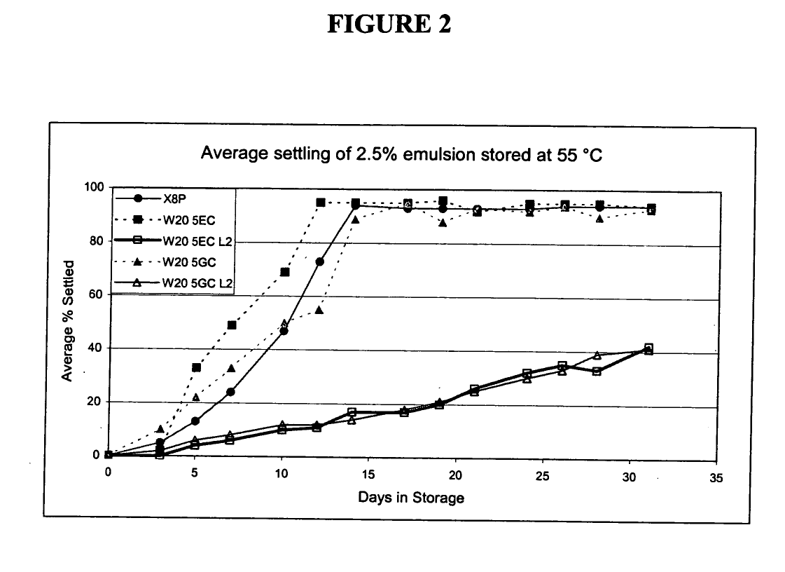

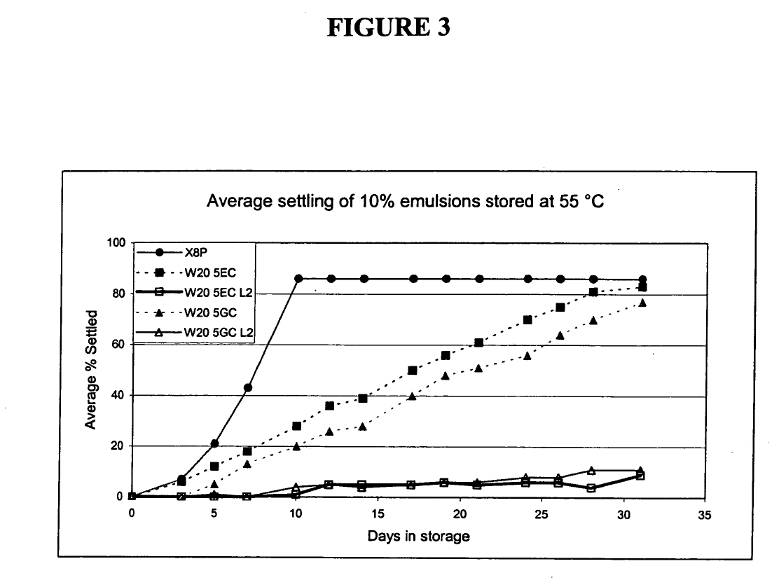

Settling and creaming are more pronounced in the diluted large particle size emulsions compared to the diluted emulsions stored at 55° C. (FIGS. 2-3, Table 12). The 10% W₂₀5EC nanoemulsion is 83% settled after 4 weeks, whereas the 10% W₂₀5EC L2 nanoemulsion is only 9% settled. The onset of settling occurred later in the smaller particle size nanoemulsion, within 10 days for 10% W₂₀5EC L2 compared to only 3 days for 10% W₂₀5EC. Table 12 shows the creaming and settling of the emulsions.

Table 12 shows the separation and settling of emulsions under accelerated aging conditions

TABLE 12

| | Separation | | Settling | | | |
|---|---|---|---|---|---|---|
| | Neat | | 10% | | 2.50% | |
| Nanoemulsion | Oil | Water | Cream | Settling | Cream | Settling |
| X8P | 9 | 17 | 13 | 86 | 6 | 94 |
| W₂₀5EC | 2 | 0 | 14 | 83 | 5 | 94 |
| W₂₀5EC L2 | 3 | 0 | 2 | 9 | 2 | 42 |
| W₂₀5EC L2 full* | 0 | 0 | 2 | <14 | 2 | 28 |
| W₂₀5GC | 0.3** | 0 | 13 | 77 | 5 | 93 |
| W₂₀5GC L2 | 0.7** | 0 | 0 | 11 | 2 | 41 |

The W₂₀ 5EC L2 nanoemulsion that is stored in vials that are completely full show no separation and less settling compared to the same nanoemulsion stored in vials containing an air space (Table 12). Interestingly, the bottom breaks off at the seam at day 10 and day 21 for 2 of the full vials of diluted nanoemulsion.

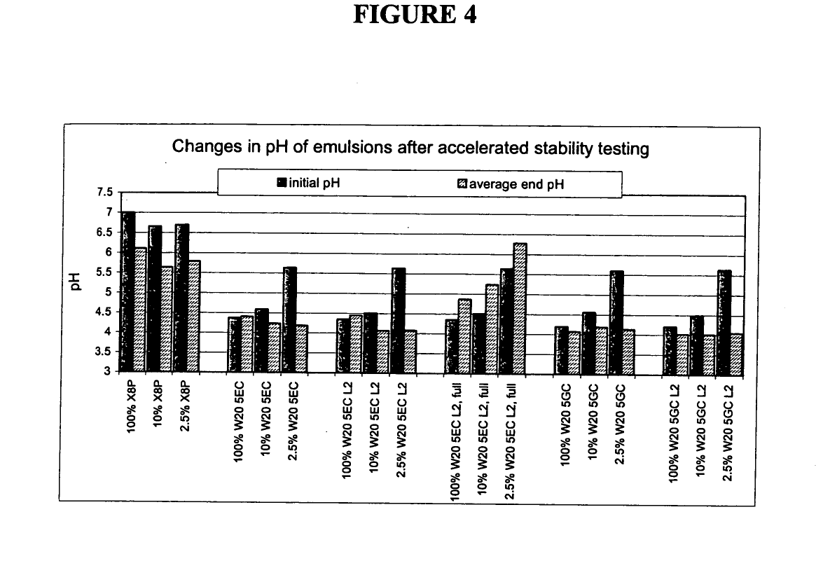

The change in pH after accelerated stability testing is measured. The pH of each nanoemulsion is measured at the beginning and at the end of the accelerated stability incubation at 55° C. Diluted emulsions are measured using a 3-in-1 combination electrode and neat emulsions are measured with a semi-micro electrode. The initial pH of the neat W₂₀5EC, and W₂₀5EC L2, W₂₀5GC, and W₂₀5GC L2 emulsions is similar for each nanoemulsion, ranging from 4.2-4.4. The pH increases with increasing dilution of these nanoemulsion to a pH of 5.6 for the 2.5% dilutions. After 4 weeks at 55° C., the pH of the neat emulsions remains unchanged, whereas the pH of the diluted emulsions decreases to a value similar to that of the neat nanoemulsion, (4.0-4.4). In contrast, W₂₀5EC L2 incubated in vials that are filled completely, slightly increased in pH after 4 weeks incubation at 55° C. The difference between the neat and diluted nanoemulsion is also maintained (FIG. 4).

Additional stress testing is preformed by centrifugation, freezing and autoclaving. In the centrifugation test, neat (100%) and a 10% dilution of W₂₀5EC L2 nanoemulsion are centrifuged at 1,650×g for 30 minutes at room temperature, then stored at room temperature for observation. An additional sample of the 10% dilution of W₂₀5EC L2 is not centrifuged and is stored at room temperature for comparison. After storage at room temperature for 6 weeks, no separation of neat or diluted emulsions is observed. Only slight creaming is seen in the 10% diluted emulsions with no difference between the centrifuged and uncentrifuged sample.

In the freezing test at −18° C. neat nanoemulsion and a 10% dilution of W₂₀5EC L2 are placed at −18° C. for 24 hours, and then left at room temperature for observation. The neat nanoemulsion W₂₀5EC L2 is frozen at −18° C. for 24 hours then thawed and observed. After 24 hours at room temperature no separation is observed in the neat or 10% diluted nanoemulsion. Creaming is observed in the 10% diluted nanoemulsion and no settling were noted.

In the autoclaving test neat W₂₀5EC, W₂₀5EC L2, W₂₀5GC, and W₂₀5GC L2 emulsions are placed in a Yamato autoclave for 15 minutes at 121° C., and then stored at room temperature for observation. Both emulsions containing ethanol (W₂₀5EC and W₂₀5EC L2) boiled over in the autoclave and severe separation is observed immediately after autoclaving. The emulsions containing glycerol are intact after autoclaving and displayed no separation up to 3 days when stored at room temperature.

### EXAMPLE 8

### Manufacture of Small Particle Size Nanoemulsions Using a High Pressure Homogenizer

This example demonstrates using a high pressure homogenizer (Avestin Emulsiflex C3) to reduce the particle size of a standard nanoemulsion to particles having a diameter of 50-150 nm. The size of the nanoemulsion particles depends on the pressure and number of passages.

First, a standard nanoemulsion containing particles having an average diameter of 250 nm to 5 micrometers, preferably about 300 nanometer to 1 micrometer is formed. The standard nanoemulsion contains 22 vol % distilled water, 1 wt/vol % cetylpyridinium chloride, 5 vol % Tween 20, 64 vol % soybean oil, 8 vol % ethanol and 2 mM EDTA, based on the total volume of the mixture formed. The nanoemulsion is formed by mixing for 5 minutes at 10,000±500 revolutions per minute with a Silverson L4RT mixer with a standard mixing assembly and a fine emulsion screen. The standard nanoemulsion is denoted as W₂₀5EC ED.

Small particle size nanoemulsions containing particles of various sizes are then formed by passing the standard nanoemulsion through an Avestin EmulsiFlex under different pressures ranging from 3,500-17,000 psi. The nanoemulsion was passed between 4-5 times under the same conditions. The machine applies high pressure to push the nanoemulsion through a dynamic homogenizing valve. Table 13 describes the different nanoemulsion particle size resulting from different passages into the emulsifier.

TABLE 13

| | Passages in the | | |
|---|---|---|---|
| | high pressure | | |
| Name | emulsifier | Pressure (psi) | Particle size (nm) |
| W₂₀5EC ED | None | — | 277 |
| W₂₀5EC ED L3 | 1 | 17,000 | 111 |
| W₂₀5EC ED L3 | 2 | 17,000 | 92 |
| W₂₀5EC ED L3 | 3 | 17,000 | 91 |
| W₂₀5EC ED L3 | 4 | 17,000 | 65 |
| W₂₀5EC ED L3 | 1 | 3,500 | 164 |
| W₂₀5EC ED L3 | 2 | 3,500 | 123 |
| W₂₀5EC ED L3 | 3 | 3,500 | 110 |
| W₂₀5EC ED L3 | 4 | 3,500 | 124 |
| W₂₀5EC ED L3 | 5 | 3,500 | 130 |

Table 13 and FIG. 5 demonstrate that particle size is inversely dependent on the amount of pressure applied during homogenization as well as the number of passages to which the nanoemulsion is subjected.

### EXAMPLE 9

### Testing of Disinfectants Containing the Nanoemulsions

Example 9 compares the efficacy of a standard nanoemulsion versus a small particle size nanoemulsion (denoted L2) as a disinfectant.

The AOAC (Association of Official Analytical Chemist) dilution test is a carrier-based test. Carriers (i.e., stainless steel cylinders) are inoculated with a test microorganism, dried, exposed to a dilution of a disinfectant product, and cultured to assess the survival of the bacteria. A single test involves the evaluation of 60 inoculated carriers contaminated with one microorganism against one product sample. In addition to the 60 carriers, 6 carriers are required to estimate carrier bacterial load and 6 more are included as extras. Thus, a total of 72 seeded carriers are required to perform a single test.

A contaminated dried cylinder carrier is added to the medication tubes. Immediately after placing carrier in medication tube, tubes are swirled 3 times before placing tube into bath. Ten minutes after each carrier is deposited into the disinfectant, each carrier is removed from the medication tube with a sterile hook, tapped against the interior sides of the tube to remove the excess disinfectant, and transferred into the primary subculture tube containing the appropriate neutralizer (Letheen broth, 10 mL in 20×150 mm tubes). The subculture tubes are swirled for 34 seconds. Transfer into the primary subculture tubes should be within ±5 seconds of the actual time of transfer (10 minutes). The bacterial carrier load on at least 2 carriers is assayed.

After a minimum of 30 minutes from when the test carrier was deposited, each carrier is transferred using a sterile wire hook to a second subculture tube containing 10 mL of the appropriate neutralizer. The carriers are transferred in order, but the intervals do not have to be timed. The tubes are swirled for 3-4 seconds and the subcultures incubated at 37° C. for 48 hours. If the broth culture appears turbid, the result is positive. A negative result is one in which the broth appears clear. Each tube is shaken prior to recording results to determine the presence or absence of turbidity. The primary and secondary subculture tubes for each carrier represent a “carrier set.” A positive result in either the primary or secondary subculture tube is considered a positive result for a carrier set.

Gram stains are performed on smears taken from the positive culture tubes. For additional confirmatory tests, a loop of broth is streaked on the selective media appropriate for the test microorganism and incubated for 24 hours at 37° C.

Table 14. Gram staining and culture on selective media required to ensure the identity of the microorganism.

TABLE 14

| | S. choleraesuis | S. aureus | P. aeruginosa |
|---|---|---|---|
| Gram stain | Gram negative | Gram positive | Gram negative |
| | rods | cocci arranged | rods |
| | | in clusters | |
| Selective media | MacConkey agar | Mannitol salt | Pseudosel agar |
| | | agar | |
| Morphology on | Pale large | Circular, small, | Circular, small, |
| selective media | colonies, agar | fluorescent | initially opaque, |
| | turning light | yellow | turning |
| | color. | colonies. | fluorescent |
| | | | green over |
| | | | time. |
| Regular media | TSA* | TSA | TSA |

*Tryptic soy agar

Table 15 show the results for a W₂₀5G BA2+2 mM EDTA at pH 7.2 nanoemulsion and a W₂₀5G BA2 L2+2 mM EDTA at pH 7.2 nanoemulsion with Staphylococcus aureus.

TABLE 15

| Sam- | | Carriers | Total | Number of | Percentage |
|---|---|---|---|---|---|
| ple | Formulation | failed | tested | experiments | failed |
| 1 | 1% W₂₀5G BA2 + | 16 | 304 | 6 | 5.26% |
| | 2 mM EDTA | | | | |
| 2 | 1% W₂₀5G BA2 L2 + | 2 | 240 | 4 | 0.83% |
| | 2 mM EDTA | | | | |
| 3 | 1% W₂₀5G BA2 L2 | 1 | 300 | 6 | 0.33% |

As shown in Table 15, a disinfectant made with the small particle size nanoemulsions has a lower failure ratio than a standard nanoemulsion. The standard nanoemulsion has a failure rate of about 5%. The small particle size nanoemulsions have a failure rate of less than 1%.

Table 16 also shows results obtained for various formulations exposed to Staphylococcus aureus.

TABLE 16

| | | | No. of | Percent- |
|---|---|---|---|---|
| Sam- | | Number of | Failed | age |
| ple | Formulation | Experiments | Cylinders | failed |
| 1 | 1% W₂₀5G BA2 + 2 mM | 6 | 304 | 5.3% |
| | EDTA pH 7.2 | | | |
| 2 | 1% W₂₀5G BA2 + 2 mM | 9 | 272 | 11.4% |
| | EDTA pH 8.0 | | | |
| 3 | 1% W₂₀5G BA2 L2 + 2 mM | 4 | 240 | 0.83% |
| | EDTA pH 7.2 | | | |
| 4 | 1% W₂₀5G BA2 pH 7.2 | 6 | 300 | 0.33% |

Table 16 demonstrates that the small particle size nanoemulsions (Samples 3 and 4) show greater efficacy against Staphylococcus aureus than the standard emulsions (Samples 1 and 2).

Table 17 shows the results obtained for various formulations exposed to Salmonella choleraesuis.

TABLE 17

| | | | No. of | Percent- |
|---|---|---|---|---|
| Sam- | | Number of | Cylinders | age |
| ple | Formulation | Experiments | tested | failed |
| 1 | 1% W₂₀5G BA2 + 2 mM | 2 | 120 | 0% |
| | EDTA pH 7.2 | | | |
| 2 | 1% W₂₀5G BA2 + 2 mM | 1 | 30 | 0% |
| | EDTA pH 8.0 | | | |
| 3 | 1% W₂₀5G BA2 L2 + 2 mM | 1 | 60 | 0% |
| | EDTA pH 7.2 | | | |
| 4 | 1% W₂₀5G BA2 (L₂) pH 7.2 | 60 | 240 | 0% |

Table 17 demonstrates that the small particle size nanoemulsions (Samples 3 and 4) show similar efficacy against Salmonella choleraesuis compared to the standard emulsions (Samples 1 and 2). Overall in the disinfectant test, the small particle size nanoemulsions perform as well as or better than the standard emulsions.

### EXAMPLE 10

### Bactericidal Properties of the Nanoemulsions Against Staphylococcus aureus

The bactericidal activity of the nanoemulsions is tested using a tube rotation test. In this test, first a culture is prepared by picking one colony from the stock culture plate of Staphylococcus aureus, streaking fresh TSA and incubating overnight at 37° C. The next morning, one colony is picked from the agar plate and transferred into 25 mL of TSB in a 50 mL screw-cap tube and incubated at 37° C. on a tube rotator for 4-5 hours until the culture becomes turbid. Bacteria grown for 4-6 hours is added to 10 mL TSB until the culture media becomes slightly turbid.

W₂₀5EC and W₂₀5EC L2 are used as previously described. The emulsions are then diluted to 2%, 1%, 0.2%, 0.1%, and 0.02% by volume with water.

Bactericidal testing is performed as follows. In 1.7 mL microfuge tubes, 0.5 mL cell suspension and 0.5 mL of each of the nanoemulsion dilutions is mixed and the tubes capped. A positive control containing 0.5 mL of cell suspension and 0.5 mL of sterile distilled water is prepared in parallel. The tubes are incubated on a tube rotator at 37° C. for 10 minutes. Each of the preparations is serially diluted (5 log diluation) in a 96-well plate using PBS. 25 μL from each dilution on is incubated on TSA at 37° C. overnight. The colonies on the control and test plates are counted. The count on the control plate provides the initial bacterial count. The initial bacteria count is provided as:
Initial bacterial count=CFU×40×plate dilution

where CFU is the colony forming units per mL. The colonies on each of the test plates is counted. Plates having between 20-50 CFU are counted. The report log reduction is provided as:
Report Log reduction=Log (count on the control treatment)−Log (count on the treatment).

The results are shown in Table 18.

TABLE 18

| | | | | | | |
|---|---|---|---|---|---|---|
| Zero | Control | 1% | 0.5% | 0.1% | 0.05% | 0.01% |

W₂₀5EC

| | | | | | | | |
|---|---|---|---|---|---|---|---|
| Log | 5 | 5 | 1 | 1 | 3 | 5 | 5 |
| Count | 193, 201 | 215, 150 | 0 | 0 | 52, 77 | 261, 236 | 225, 237 |
| % Kill | | 7.36 | 100.00 | 100.00 | 99.67 | −26.14 | −17.26 |
| Log R. | | 0.03 | 6.29 | 6.29 | 2.48 | −0.10 | −0.07 |

W₂₀5EC L2

| | | | | | | | |
|---|---|---|---|---|---|---|---|
| Log | 5 | 5 | 1 | 1 | 4 | 5 | 5 |
| Count | 146, 129 | 167, 184 | 0, 0 | 0, 0 | 289, 246 | 196, 206 | 149, 170 |
| % Kill | | −27.64 | 100.00 | 100.00 | 80.55 | −46.18 | −16.00 |
| Log R. | | −0.11 | 6.14 | 6.14 | 0.71 | −0.16 | −0.06 |

Note:
a (—) log killing is considered zero.

As shown in Table 19, at 0.01% and 0.05% dilution, neither the standard nanoemulsion nor the small particle size nanoemulsion has a significant effect on the viability of the S. aureus. The 1%, and 0.5% dilutions, however, have similar effects on S. aureus viability, with 100% killing at 1% and 0.5% for both particle sizes. The 0.1% dilutions show slightly better killing in the nanoemulsion compared with the small particle size nanoemulsion.

Small particle size nanoemulsions have several advantages over standard emulsions. First, the small particle size nanoemulsions can be more stable than the standard emulsions when stored at room temperature or at 55° C. The small particle size nanoemulsions are capable of resisting separation or settling when stored at room temperature for four months. The undiluted small particle size nanoemulsions can take about 1 to 3 days longer to exhibit moderate separation than the standard emulsions. The 2.5% to 10% diluted small particle size nanoemulsions can take about 2 to 7 days longer to exhibit moderate to extreme settling than the standard emulsions. In addition, the onset of phase separation in the small particle size nanoemulsions at 55° C. is later than for the standard emulsions.

Second, the small particle size nanoemulsions perform equal to or better than standard emulsions in inactivating bacteria. In a disinfectant test, the small particle size nanoemulsions exhibit a less than 1% failure rate against Staphylococcus aureus compared to greater than 5% for a standard nanoemulsion. In the same test, both the and standard nanoemulsions have a 0% failure rate against Salmonella choleraesuis. In a tube rotation test, the small particle size nanoemulsions have a slightly improved killing compared with the standard emulsions against Staphylococcus aureus killing activity.

While the invention is described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention. All references and publications cited herein are incorporated by reference in their entireties. Unless otherwise specified, “a” or “an” means “one or more.”

What is claimed is:

1. A composition comprising a nanoemulsion and having anti-inflammatory activity, the nanoemulsion comprising:
an aqueous phase;
an oil phase comprising an oil and an organic solvent;
at least one anti-inflammatory agent;
one or more surfactants; and
wherein the nanoemulsion comprises nanoemulsion particles having an average diameter of less than or equal to about 250 nm.

2. The composition of claim 1, wherein the nanoemulsion particles have an average diameter of less than or equal to about 200 nm, less than or equal to about 150 nm, less than or equal to about 100 nm, or less than or equal to about 50 nm.

3. The composition of claim 1, wherein the anti-inflammatory agent is a steroid or a non-steroidal anti-inflammatory drug.

4. The composition of claim 3, wherein the steroid is selected from the group consisting of dipropionate (Diprolene), clobetasol 17-Propionate (Dermovate), halobetasolpropionate (Ultravate), Halcinonide (Halog), amcinonide (Cyclocort), betamethasone dipropionate (Diprolene, generics), betamethasone valerate (Betaderm, Belestoderm,Prevex), Desoximetasone (Desoxi,Topicort), diflucortolone valerate (Nerisone), fluocinonlone acetonide (Derma,Fluoderm,Synalar), fluocinonide (Lidemol, Lidex, Tyderm, Tiamol, Topsyn), mometasone furoate, betamethasone valerate (Betnovate), betamethasone valerate (Celestoderm), clobetasone 17-butyrate (Eumovate), desonide (Desocort), hydrocortisone acetate (Cortef, Hyderm),hydrocortisone valerate (Westcort, Hydroval), prednicarbate (Dermatop), triamcinolone acetonide (Kenalog,Traiderm), loratodine (Claratin) desonide (Desocort), hydrocortisone (Cortate, Cortoderm), hydrocortisone acetate (Cortef, Hyderm), and a combination thereof.

5. The composition of claim 1, wherein the non-steroidal anti-inflammatory drug is selected from the group consisting of aspirin (Anacin, Ascriptin, Bayer, Bufferin, Ecotrin, Excedrin), choline and magnesium salicylates (CMT, Tricosal, Trilisate), choline salicylate (Arthropan), celecoxib (Celebrex), diclofenac potassium (Cataflam),diclofenac sodium (Voltaren, Voltaren XR), diclofenac sodium with misoprostol (Arthrotec), diflunisal (Dolobid), etodolac (Lodine, Lodine XL), fenoprofen calcium (Nalfon), flurbiprofen (Ansaid), ibuprofen (Advil, Motrin, Motrin IB, Nuprin), indomethacin (Indocin, Indocin SR), ketoprofen (Actron, Orudis, Orudis KT, Oruvail), magnesium salicylate (Arthritab, Bayer Select, Doan's Pills, Magan, Mobidin, Mobogesic), meclofenamate sodium (Meclomen), mefenamic acid (Ponstel), meloxicam (Mobic), nabumetone (Relafen), naproxen (Naprosyn, Naprelan), naproxen sodium (Aleve, Anaprox), oxaprozin (Daypro), piroxicam (Feldene), rofecoxib (Vioxx), salsalate (Amigesic, Anaflex 750, Disalcid, Marthritic, Mono-Gesic, Salflex, Salsitab), sodium salicylate, sulindac (Clinoril), tolmetin sodium (Tolectin), valdecoxib (Bextra), and a combination thereof.

6. The composition of claim 1, wherein the organic solvent comprises a C₁-C₁₂ alcohol, diol, or triol, a dialkyl phosphate, a trialkyl phosphate or a combination thereof.

7. The composition of claim 1, wherein the alcohol comprises ethanol, isopropyl alcohol, glycerol or a combination thereof.

8. The composition of claim 6, wherein the trialkyl phosphate is tri-n-butyl phosphate.

9. The composition of claim 1, wherein the oil comprises soybean oil, mineral oil, avocado oil, squalene oil, olive oil, canola oil, corn oil, rapeseed oil, safflower oil, sunflower oil, fish oils, flavor oils, cinnamon bark, coconut oil, cottonseed oil, faxseed oil, pine needle oil, silicon oil, essential oils, water insoluble vitamins, or a combination thereof.

10. The composition of claim 9, wherein the oil comprises soybean oil.

11. The composition of claim 1, wherein the surfactant is a nonionic surfactant.

12. The composition of claim 11, wherein the nonionic surfactant is TWEEN® 20, Triton® X-100, nonoxynol-9, or a combination thereof.

13. The composition of claim 1, wherein the surfactant is a cationic surfactant.

14. The composition of claim 13, wherein the cationic surfactant is cetyl pyridimium chloride, benzalkonium chloride or a combination thereof.

15. The composition of claim 1 comprising:
about 5 vol. % to about 50 vol. % of aqueous phase;
about 30 vol. % to about 90 vol. % of oil phase; and
about 3 vol. % to about 15 vol. % of surfactant.

16. The composition of claim 1, further comprising an additive selected from the group consisting of activity modulators, gelling agents, auxiliary surfactants, and a combination comprising one or more of the foregoing additives.

17. The composition of claim 16, wherein the activity modulator is an interaction enhancer, a germination enhancer, a therapeutic agent, or a combination comprising one or more of the foregoing enhancers.

18. The composition of claim 16, wherein the germination enhancer comprises glucose, fructose, asparagine, sodium chloride, ammonium chloride, calcium chloride, and potassium chloride.

19. The composition of claim 17, wherein the germination enhancer comprises L-alanine, inosine, PBS, and ammonium chloride.

20. The composition of claim 17, wherein the therapeutic agent is an antimicrobial agent, an antifungal agent, an antiviral agent, an anti-mold agent, an anti-mildew agent, or a combination thereof.

21. The composition of claim 17, wherein the therapeutic agent is a penicillin, a cephalosporin, cycloserine, vancomycin, bacitracin, miconazole, ketoconazole, clotrimazole, polymyxin, colistimethate, nystatin, amphotericin B, chloramphenicol, the tetracyclines, erythromycin, clindamycin, an aminoglycoside, a rifamycin, a quinolone, trimethoprim, a sulfonamide, zidovudine, gangcyclovir, vidarabine, acyclovir, phenylphenol, propyl paraben, poly(hexamethylene biguanide) or a combination comprising one or more of the foregoing therapeutic agents.

22. The composition of claim 1, wherein the composition comprises from about 0.01% to about 90% nanoemulsion per milliliter of composition.

23. The composition of claim 1, wherein the composition comprises greater than about 0.25%, about 1.0%, about 5%, about 10%, about 20%, about 35%, about 50%, about 65%, about 80%, about 90%, or about 95% nanoemulsion per milliliter of composition.

24. The composition of claim 1, further comprising a pharmaceutically acceptable carrier, an auxiliary surfactant, a suds suppressor, a detergent builder, or a combination thereof.

25. A method of reducing the average nanoemulsion particle size of a composition having anti-inflammatory activity that comprises a nanoemulsion, comprising treating a nanoemulsion comprising an aqueous phase, an oil phase comprising an oil and an organic solvent, and a surfactant, and having nanoemulsion particles of an average diameter of greater than or equal to about 250 nm, so as to reduce the average diameter of the nanoemulsion particles to less than or equal to about 250 nm.

26. The method of claim 25, wherein the nanoemulsion particles are reduced to an average diameter of less than or equal to about 200 nm, less than or equal to about 150 nm, less than or equal to about 100 nm, or less than or equal to about 50 nm.

27. The method of claim 25, wherein the anti-inflammatory agent is a steroid or a non-steroidal anti-inflammatory drug.

28. The method of claim 27, wherein the steroid is selected from the group consisting of dipropionate (Diprolene), clobetasol 17-Propionate (Dermovate), halobetasolpropionate (Ultravate), Halcinonide (Halog), amcinonide (Cyclocort), betamethasone dipropionate (Diprolene, generics), betamethasone valerate (Betaderm, Belestoderm,Prevex), Desoximetasone (Desoxi,Topicort), diflucortolone valerate (Nerisone), fluocinonlone acetonide (Derma,Fluoderm,Synalar), fluocinonide (Lidemol, Lidex, Tyderm, Tiamol, Topsyn), mometasone furoate, betamethasone valerate (Betnovate), betamethasone valerate (Celestoderm), clobetasone 17-butyrate (Eumovate), desonide (Desocort), hydrocortisone acetate (Cortef, Hyderm),hydrocortisone valerate (Westcort, Hydroval), prednicarbate (Dermatop), triamcinolone acetonide (Kenalog,Traiderm), loratodine (Claratin) desonide (Desocort), hydrocortisone (Cortate, Cortoderm), and hydrocortisone acetate (Cortef, Hyderm).

29. A method of making a nanoemulsion having anti-inflammatory activity, comprising
passing a first nanoemulsion comprising
an aqueous phase,
an oil phase comprising an oil and an organic solvent,
at least on anti-inflammatory agent, and
one or more surfactants, wherein the nanoemulsion particles have an average diameter of greater than or equal to about 250 nm through a high pressure homogenizer or a microfluidizer under conditions effective to reduce the average diameter of the nanoemulsion particles less than or equal to about 250 nm.

30. The method of claim 29, wherein the nanoemulsion particles are reduced to an average diameter of less than or equal to about 200 nm, less than or equal to about 150 nm, less than or equal to about 100 nm, or less than or equal to about 50 nm.

31. The method of claim 25, wherein the ratio of oil phase to aqueous phase is from about 1:9 to about 5:1, from about 5:1 to about 3:1, or from about 4:1.

32. The method of claim 29, wherein the ratio of oil phase to aqueous phase is from about 1:9 to about 5:1, from about 5:1 to about 3:1, or from about 4:1.

33. A method of inactivating a microorganism, comprising contacting the microorganism with a composition comprising a nanoemulsion and having anti-inflammatory activity for a time effective to inactivate the microorganism, wherein the nanoemulsion comprises:
an aqueous phase;
an oil phase comprising an oil and an organic solvent;
at least one anti-inflammatory agent
and one or more surfactants;
and wherein the nanoemulsion particles have an average diameter of less than or equal to about 250 nm.

34. The method of claim 33, wherein the nanoemulsion comprises nanoemulsion particles having an average diameter of less than or equal to about 200 nm, less than or equal to about 150 nm, less than or equal to about 100 nm, or less than or equal to about 50 nm.

35. The method of claim 33, wherein the composition further comprises an activity modulator, wherein the activity modulator is an interaction enhancer, a germination enhancer, a therapeutic agent, or a combination comprising one or more of the foregoing.

36. The method of claim 33, wherein the composition further comprises a pharmaceutically acceptable carrier.

37. The method of claim 33, wherein the microorganism is a bacteria, a fungus, a protozoa, a virus, or a combination of one or more of the foregoing microorganisms.

38. The method of claim 37, wherein the bacteria is a vegetative bacteria, a bacterial spore, or a combination thereof.

39. The method of claim 37, wherein the bacteria comprises a Gram negative bacteria, a Gram positive bacteria, an acid fast bacilli, or a combination thereof.

40. The method of claim 38, wherein the bacterial spore is B. anthracis.

41. The method of claim 37, wherein the bacteria comprises comprises B. anthracis, B. cereus, B. circulans, B. megatertium, B. subtilis, C. botulinum, C. tetani, C. perfringens, H. influenzae, N. gonorrhoeae, S. agalactiae, S. pneumonia, S. pyogenes, V. cholerae, S. aureus, Yersinia species, G. vaginalis, G. mobiluncus, M. hominis, Salmonellae species, Shigellae species, Pseudomonas species, Eschericia species, Klebsiella species, Proteus species, Enterobacter species, Serratia species, Moraxella species, Legionella species, Bordetella species, Helicobacter species, Arthobacter species, Micrococcus species, Listeria species, Corynebacteria species, Planococcus species, Nocardia species, Rhodococcus species, Mycobacteria species, or a combination thereof.

42. The method of claim 37, wherein the virus belongs to a family selected from the group consisting of Orthomyxoviridae, Retroviridae, African Swine Fever Viruses, Papovaviridae, Hepadnaviridae, Coronaviridae, Flaviviridae, Togaviridae, Picornaviridae, Filoviridae, Paramyxoviridae, or Rhabdoviridae.

43. The method of claim 42, herein the Orthomyxovirdae virus is influenza virus, herpes simplex, herpes zoster, sendai virus, sindbis virus, pox virus, small pox or vaccinia virus.

44. The method of claim 42, herein the Retroviridae is human immunodeficiency virus, west nile virus, hanta virus, or human papilloma virus.

45. The method of claim 37, wherein the fungus is a yeast or a filamentous fungus.

46. The method of claim 37, wherein filamentous fungus is selected from the group consisting of an Aspergillus species or a dermatophyte.

47. The method of claim 46 wherein the dermatophyte is selected from the group consisting of Trichophyton rubrum, Trichophyton mentagrophytes, Microsporum canis, Microsporum gypseum and Epidermophyton floccosum.

48. The method of claim 37 wherein molds comprises Cladosporium, Fusarium, Alternaria, Curvularia, Aspergiflus and Penicillium.

49. A method of inactivating a pathogenic microorganism comprising contacting a subject infected with the microorganism with a composition having anti-inflammatory activity comprising a nanoemulsion comprising:
an aqueous phase;
an oil phase comprising an oil and an organic solvent;
at least one anti-inflammatory agent; and
one or more surfactants;
wherein the nanoemulsion comprises particles having an average diameter of less than or equal to about 250 nm.

50. The method of claim 49, wherein the particles have an average diameter of less than or equal to about 200 nm, less than or equal to about 150 nm, less than or equal to about 100 nm, or less than or equal to about 50 nm.

51. The method of claim 49, wherein the subject is a human, an animal, or a plant.

52. The method of claim 49, wherein the composition further comprises an activity modulator, wherein the activity modulator is an interaction enhancer, a germination enhancer, a therapeutic agent, or a combination comprising one or more of the foregoing.

53. The method of claim 49, wherein the composition further comprises a pharmaceutically acceptable carrier.

54. The method of claim 49, wherein the microorganism is a bacteria, a fungus, a protozoa, a virus, or a combination of one or more of the foregoing microorganisms.

55. The method of claim 54, wherein the bacteria is a vegetative bacteria, a bacterial spore, or a combination thereof.

56. The method of claim 54, wherein the fungus is a yeast.

57. The method of claim 56, wherein the bacteria comprises a Gram negative bacteria, a Gram positive bacteria, an acid fast bacilli, or a combination thereof.

58. The method of claim 56, wherein the bacterial spore is B. anthracis.

59. The method of claim 54, wherein the bacteria is B. anthracis, B. cereus, B. circulans, B. megatertium, B. subtilis, C. botulinum, C. tetani, C. perfringens, H. influenzae, N. gonorrhoeae, S. agalactiae, S. pneumonia, S. pyogenes, V. cholerae, S. aureus, Yersinia species, G. vaginalis, G. mobiluncus, M. hominis, Salmonellae species, Shigellae species, Pseudomonas species, Eschericia species, Kiebsiella species, Proteus species, Enterobacter species, Serratia species, Moraxella species, Legionella species, Bordetella species, Helicobacter species, Arthobacter species, Micrococcus species, Listeria species, Corynebacteria species, Planococcus species, Nocardia species, Rhodococcus species, Mycobacteria species, or a combination thereof.

60. The method of claim 55, wherein the virus belongs to a family selected from the group consisting of Orthomyxoviridae, Retroviridae, African Swine Fever Viruses, Papovaviridae, Hepadnaviridae, Coronaviridae, Flaviviridae, Togaviridae, Picornaviridae, Filoviridae, Paramyxoviridae, or Rhabdoviridae.

61. The method of claim 60, herein the Orthomyxovirdae virus is influenza virus, herpes simplex, herpes zoster, sendai virus, sindbis virus, pox virus, small pox or vaccinia virus.

62. The method of claim 60, herein the Retroviridae is human immunodeficiency virus, west nile virus, hanta virus, or human papilloma virus.

63. The method of claim 56, wherein filamentous fungus comprises Aspergillus species or dermatophytes such as Trichophyton rubrum, Trichophyton mentagrophytes, Microsporum canis, Microsporum gypseum and Epidermophyton floccosum.

64. The method of claim 54 wherein the fungus is selected from the group consisting of Cladosporium, Fusarium, Alternaria, Curvularia, Aspergillus and Penicillium.

65. A method of preventing an infected state caused by a microorganism, comprising administering to a subject, either before or after exposure to a microorganism, a composition having anti-inflammatory activity comprising a nanoemulsion, wherein the nanoemulsion comprises:
an aqueous phase;
an oil phase comprising an oil and an organic solvent;
at least one anti-inflammatory agent; and
one or more surfactants;
wherein the nanoemulsion comprises particles having an average diameter of less than or equal to about 250 nm.

66. The method of claim 65, wherein the infected state is a sexually transmitted genital infection.

67. The method of claim 66, wherein the sexually transmitted genital infection is selected from the group consisting of genital herpes, human papilloma virus, human immunodeficiency virus, trichomoniasis, gonorrhea, syphilis, and Chlamydia.

68. The method of claim 65, wherein the microorganism is selected from the group consisting of a pox virus, B. anthracis, and Yersinia species,

69. The method of claim 65, wherein the step of administering comprises application of the composition to the mucosa of a subject.

70. A kit comprising a composition having anti-inflammatory activity comprising a nanoemulsion, wherein the composition is provided in a single formulation or a binary formulation, wherein the binary formulation is mixed prior to using the composition.

71. The kit of claim 70, further comprising instructions for using the composition.

72. The kit of claim 71, wherein the composition is formulated for topical administration, said kit further including means for applying the composition topically.
